(19) United States
(12) Patent Application Publication
(10) Pub. No.: US 2020/0016573 A1
(43) Pub. Date: Jan. 16, 2020

(54) ABSORBENT COMPOSITE, INK ABSORBING MATERIAL, DEODORANT, DEODORIZER, AND COSMETICS

(71) Applicant: SEIKO EPSON CORPORATION, Tokyo (JP)

(72) Inventors: Shinobu YOKOKAWA, Okaya (JP); Yoichi MIYASAKA, Suwa (JP); Kaneo YODA, Okaya (JP); Nobutaka URANO, Matsumoto (JP)

(21) Appl. No.: 16/507,216

(22) Filed: Jul. 10, 2019

(30) Foreign Application Priority Data
Jul. 12, 2018 (JP) 2018-132112

(51) Int. Cl. B01J 20/26; A61K 8/81; A61Q 19/00; A61Q 5/00; A61K 8/02; B01J 20/28; A61L 9/014

(52) U.S. Cl. CPC B01J 20/267; A61K 8/8176; A61Q 19/00; A61K 2800/10; A61K 8/0208; B01J 20/28028; A61L 9/014; A61Q 5/00

(57) ABSTRACT

An absorbent composite includes a water absorbent resin in which a nonionic cross-linked polymer and an anionic cross-linked polymer are mixed, and a fiber base material containing a fiber, and a content of the water absorbent resin in the absorbent composite is 5% or more and less than 65%.

The present application is based on, and claims priority from JP Application Serial Number 2018-132112, filed Jul. 12, 2018, the disclosure of which is hereby incorporated by reference herein in its entirety.

### BACKGROUND

### 1. Technical Field

The present disclosure relates to an absorbent composite, an ink absorbing material, a deodorant, a deodorizer, and cosmetics.

### 2. Related Art

In the related art, an absorbent composites is known which includes a base material and water absorbent resin particles, and in which a weight ratio of the water absorbent resin to the total weight of the base material and the water absorbent resin is 65% to 94% by weight (for example, European Patent No. 1880842).

However, when the weight ratio of the water absorbent resin to the total weight of the base material and the water absorbent resin is 65% by weight or more, the water absorbent resin particles are likely to be detached from the base material. In addition, in an absorbent composite using one type of water absorbent resin, for example, there is a problem that it is difficult to absorb both a pigment-based ink containing a pigment and a dye-based ink containing a dye in an ink absorbing material application.

### SUMMARY

According to an aspect of the present disclosure, there is provided an absorbent composite including a water absorbent resin in which a nonionic cross-linked polymer and an anionic cross-linked polymer are mixed, and a fiber base material containing a fiber, in which a content of the water absorbent resin in the absorbent composite is 5% or more and less than 65%.

In the absorbent composite, a content of the anionic cross-linked polymer may be greater than that of the nonionic cross-linked polymer in the water absorbent resin.

In the absorbent composite, a content of the nonionic cross-linked polymer may be greater than that of the anionic cross-linked polymer in the water absorbent resin.

In the absorbent composite, a content of the anionic cross-linked polymer in the water absorbent resin may be 10% or more and less than 78%.

In the absorbent composite, the water absorbent resin may be disposed between two fiber base materials in the absorbent composite.

In the above absorbent composite, the anionic cross-linked polymer may be disposed between first and second fiber base materials, and the nonionic cross-linked polymer may be disposed on a surface of the first fiber base material opposite to a side on which the anionic cross-linked polymer is disposed.

In the absorbent composite, the nonionic cross-linked polymer may be disposed on a surface of the second fiber base material opposite to the side on which the anionic cross-linked polymer is disposed.

In the absorbent composite, an amount of the nonionic cross-linked polymer disposed on any one of the surface of the first fiber base material and the surface of the second fiber base material may be greater than an amount of the anionic cross-linked polymer disposed between the first and second fiber base materials.

According to another aspect of the present disclosure, there is provided an ink absorbing material including a plurality of the above absorbent composites.

According to still another aspect of the present disclosure, there is provided a deodorant including a plurality of the above absorbent composites.

According to still another aspect of the present disclosure, there is provided a deodorizer including the above deodorant.

According to still another aspect of the present disclosure, there is provided cosmetics including a plurality of the above absorbent composites.

### BRIEF DESCRIPTION OF THE DRAWINGS

FIG. 1 is a perspective view illustrating a configuration of an absorbent composite according to a first embodiment.

FIG. 2 is a cross-sectional view illustrating the configuration of the absorbent composite according to the first embodiment.

FIG. 3 is a perspective view illustrating a configuration of an ink absorbing material according to the first embodiment.

FIG. 4 is a schematic view illustrating a method of manufacturing the absorbent composite according to the first embodiment.

FIG. 5 is a schematic view illustrating a method of manufacturing the absorbent composite according to the first embodiment.

FIG. 6 is a schematic view illustrating a method of manufacturing the absorbent composite according to the first embodiment.

FIG. 7 is a schematic view illustrating a method of manufacturing the absorbent composite according to the first embodiment.

FIG. 8 is a schematic diagram illustrating an example of a usage aspect of the ink absorbing material according to the first embodiment.

FIG. 9 is a cross-sectional view illustrating a configuration of an absorbent composite according to a second embodiment.

FIG. 10 is a schematic view illustrating a method of manufacturing the absorbent composite according to the second embodiment.

FIG. 11 is a schematic view illustrating a method of manufacturing the absorbent composite according to the second embodiment.

FIG. 12 is a schematic view illustrating a method of manufacturing the absorbent composite according to the second embodiment.

FIG. 13 is a cross-sectional view illustrating a configuration of an absorbent composite according to a third embodiment.

FIG. 14 is a schematic view illustrating a method of manufacturing the absorbent composite according to the third embodiment.

FIG. 15 is a schematic view illustrating a method of manufacturing the absorbent composite according to the third embodiment.

FIG. 16 is a schematic view illustrating a method of manufacturing the absorbent composite according to the third embodiment.

FIG. 17 is a schematic view illustrating a method of manufacturing the absorbent composite according to the third embodiment.

FIG. 18 is a schematic view illustrating an example of an aspect of an ink absorbing material according to a fourth embodiment.

FIG. 19 is a schematic view illustrating an example of an aspect of an ink absorbing material according to a fifth embodiment.

FIG. 20 is a schematic view illustrating an example of an aspect of an ink absorbing material according to a sixth embodiment.

FIG. 21 is a plan view illustrating a state of the ink absorbing material illustrated in FIG. 20 in a container.

FIG. 22 is a cross-sectional view taken along line XXII-XXII in FIG. 21.

FIG. 23 is a cross-sectional view taken along line XXIII-XXIII in FIG. 21.

FIG. 24 is a schematic view illustrating an example of a storage aspect of the ink absorbing material stored in the container.

FIG. 25 is a schematic view illustrating another configuration of an absorbent composite according to a seventh embodiment.

FIG. 26 is a schematic view illustrating another configuration of an absorbent composite according to an eighth embodiment.

FIG. 27 is a schematic view illustrating another configuration of an absorbent composite according to a ninth embodiment.

FIG. 28 is a perspective view illustrating a configuration of another ink absorber according to a tenth embodiment.

### DESCRIPTION OF EXEMPLARY EMBODIMENTS

Hereinafter, embodiments of the present disclosure will be described with reference to the drawings. In each of the following drawings, the scale of each member or the like is different from the actual scale in order to make each member or the like to be recognizable.

### First Embodiment

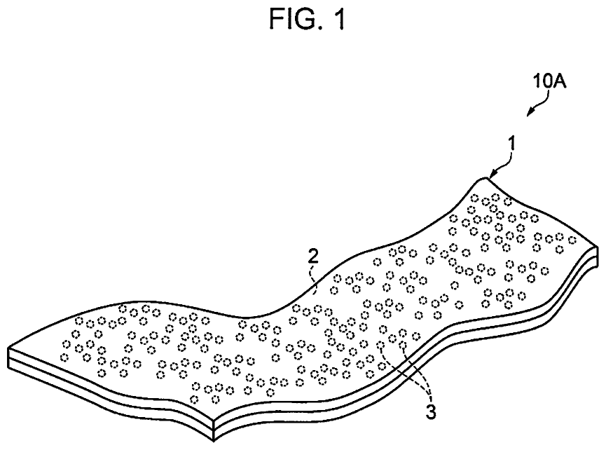

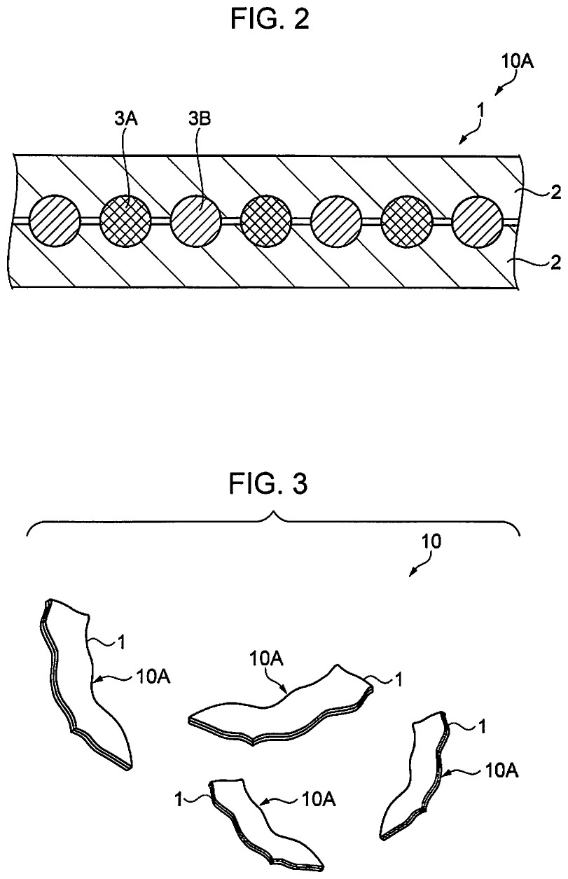

FIG. 1 is a perspective view illustrating a configuration of an absorbent composite 10A. FIG. 2 is a cross-sectional view illustrating the configuration of the absorbent composite 10A. FIG. 3 is a perspective view illustrating the configuration of an ink absorbing material 10.

The absorbent composite 10A absorbs, for example, a material containing water, such as ink.

As illustrated in FIGS. 1 and 2, the absorbent composite 10A includes a paper piece 1 having a water absorbent resin 3 in which at least a nonionic cross-linked polymer 3A and an anionic cross-linked polymer 3B are mixed, and a fiber base material 2 containing fibers (for example, pulp fibers). The fiber base material 2 may be unused paper, but in the present embodiment, recycled paper or printed waste paper is used. The majority of the absorbent composite 10A is in the form of a strip having a substantially rectangular shape in a plan view. In order to facilitate the description of the water absorbent resin 3, the nonionic cross-linked polymer is denoted as 3A and the anionic cross-linked polymer as 3B. In addition, in FIG. 2, in order to facilitate the description, the nonionic cross-linked polymer 3A and the anionic cross-linked polymer 3B are alternately arranged and represented.

Since the absorbent composite 10A is formed on the paper piece 1, for example, when ink is applied to the ink absorbing material 10 provided with a plurality of paper pieces 1, as illustrated in FIG. 3, rather than forming the ink absorbing material 10 with the fiber base material 2 formed of plate-like (or sheet-like) blocks, in a state where many opportunities for contact between the paper piece 1 and the ink can be ensured, and large contact area between the ink and the paper piece 1 can be ensured, the fibers (fiber base material 2) temporarily hold the ink. Thereafter, the ink can be efficiently fed from the fibers to the water absorbent resin 3, and absorption characteristics of the ink as the entire ink absorbing material 10 can be improved.

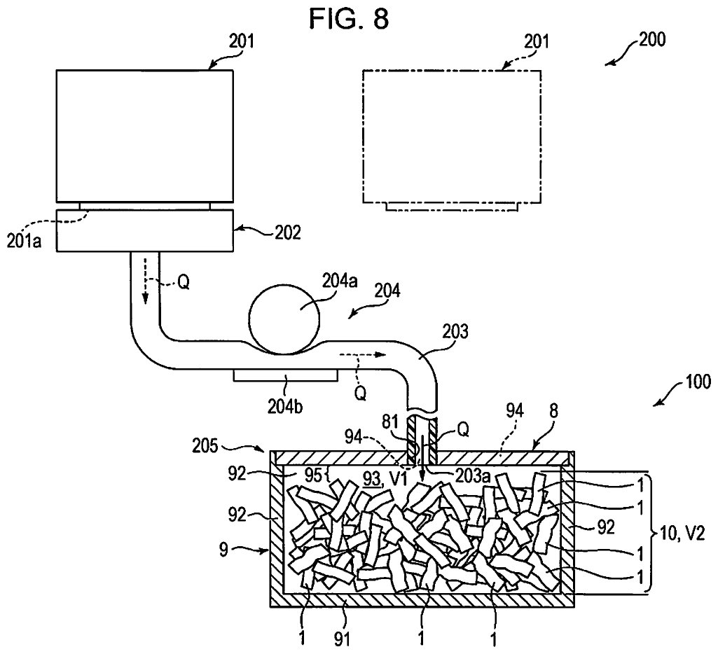

In addition, by forming the ink absorbing material 10 with the ink absorbing material 10 including a plurality of paper pieces 1, the ink absorbing material 10 can follow a predetermined shape of a container 9 described later and can store a desired amount (appropriate amount) (refer to FIG. 8). For example, adjustment of bulk density can be easily performed. As a result, it is possible to prevent the occurrence of unevenness in the absorption characteristics of the ink.

In the absorbent composite 10A of the present embodiment, the water absorbent resin 3 (nonionic cross-linked polymer 3A, anionic cross-linked polymer 3B) is disposed between the two fiber base materials 2. Since the water absorbent resin 3 is interposed between the two fiber base materials 2, it is possible to suppress the detachment of the water absorbent resin 3 from the fiber base material 2. In addition, the ink or the like permeated from the fiber base material 2 side can be absorbed and held by the water absorbent resin 3 disposed between the two fiber base materials 2.

Furthermore, the content of the water absorbent resin 3 in the absorbent composite 10A is 5% or more and less than 65%. Therefore, the contact with the fiber base material 2 and the water absorbent resin 3 is reliably performed. That is, when the content of the water absorbent resin 3 is excessive, the water absorbent resin 3 not in contact with a surface of the fiber base material 2 is increased. As a result, the water absorbent resin 3 not in contact with the surface of the fiber base material 2 is likely to be detached from the fiber base material 2. Therefore, in the present embodiment, by appropriately setting the content of the water absorbent resin 3 in the absorbent composite 10A, the fiber base material 2 and the water absorbent resin 3 can be reliably brought into contact with each other, and the detachment of the water absorbent resin 3 from the fiber base material 2 can be prevented. As a result, the absorption characteristics of the ink or the like can be exhibited over a long period of time. In addition, the water absorbent resin 3 can be prevented from detaching in the container 9. Therefore, the water absorbent resin 3 can be prevented from being unevenly distributed in the container 9. As a result, it is possible to prevent the occurrence of unevenness in the absorption characteristics of the ink.

In the present specification, “water absorption” refers to absorption applied to a wide variety of fields such as hygiene field such as hygiene products such as diapers and sanitary products, medical field, civil engineering and building field, food field, industrial field, soil conditioner, agriculture and horticulture field, and the like. In the present embodiment, the ink absorbing material 10 will be described below as an example. It refers to absorbing not only an aqueous ink in which a coloring material is dissolved in an aqueous solvent but also general ink such as a solvent-based ink in which a binder is dissolved in a solvent, a Uv curable ink in which the binder is dissolved in a liquid monomer which is cured by UV irradiation, a latex ink in which the binder is dispersed in a dispersion medium, or the like.

The fiber base material 2 contains fibers. The water absorbent resin 3 can be suitably carried by the fiber base material 2, and the detachment of the water absorbent resin 3 from the fiber base material 2 can be more suitably prevented. In addition, when the ink is applied to the paper piece 1, the fiber (fiber base material 2) temporarily holds the ink, and thereafter the ink can be efficiently fed by the water absorbent resin 3, and the absorption characteristics of the ink as the entire paper piece 1 can be improved. In addition, in general, fibers such as cellulose fibers (in particular, fibers derived from waste paper) are inexpensive than the water absorbent resin 3, and are also advantageous from the viewpoint of reducing the manufacturing cost of the paper piece 1. In addition, it is also advantageous from the viewpoint of waste reduction and effective use of resources.

Examples of the fibers include synthetic resin fibers such as polyester fibers and polyamide fibers; natural resin fibers such as cellulose fibers, keratin fibers and fibroin fibers, and chemically modified products thereof, or the like, and these may be used alone or in appropriate mixtures. It is preferable to use mainly cellulose fibers, and it is more preferable that substantially all of the fibers are cellulose fibers.

Since cellulose is a material having a suitable hydrophilic property, when ink is applied to the paper piece 1, the cellulose can be suitably fed to the water absorbent resin 3 by diffusing the ink into cellulose fibers. As a result, it is possible to make the ink absorption characteristics of the entire paper piece 1 particularly excellent. In addition, since the cellulose normally has high affinity with the water absorbent resin 3, the water absorbent resin 3 can be more suitably carried on the surface of the fiber. In addition, the cellulose fiber is a renewable natural material, and among various types of fibers, it is inexpensive and easily available, so that it is also advantageous from the viewpoints of reduction of production cost of paper piece 1, stable production, reduction of environmental load, and the like.

In the present specification, the cellulose fiber may be any fiber having cellulose as a compound (cellulose in narrow sense) as the main component and having a fibrous shape, and may contain hemicellulose and lignin in addition to cellulose (cellulose in narrow sense).

The average length of the fibers is not particularly limited, and is preferably 0.1 mm or more and 7 mm or less, more preferably 0.1 mm or more and 5 mm or less, and still more preferably 0.1 mm or more and 3 mm or less. The average width (diameter) of the fibers is not particularly limited, and is preferably 0.05 mm or more and 2 mm or less, and more preferably 0.1 mm or more and 1 mm or less.

The average aspect ratio (ratio of average length to average width) of the fibers is not particularly limited, and is preferably 10 or more and 1,000 or less, and more preferably 15 or more and 500 or less.

With the above numerical range, it is possible to more suitably carry the water absorbent resin 3, hold the ink by the fibers, and feed the ink into the water absorbent resin 3, and it is possible to make the ink absorption characteristics of the entire paper piece 1 more excellent.

The water absorbent resin 3 contains the nonionic cross-linked polymer 3A and the anionic cross-linked polymer 3B. The anionic cross-linked polymer 3B has a property of absorbing a material having a low electrolyte concentration (for example, pigment-based ink containing pigment). On the other hand, the anionic cross-linked polymer 3B has a reduced absorption property for a material having a high electrolyte concentration (for example, dye-based ink containing dye).

On the other hand, the nonionic cross-linked polymer 3A does not depend on the electrolyte concentration, and has the property of absorbing even a material with a high electrolyte concentration. On the other hand, when the nonionic cross-linked polymer 3A is compared with the anionic cross-linked polymer 3B in terms of the absorbed amount per unit weight, the absorbed amount of the nonionic cross-linked polymer 3A is as small as approximately ⅕ to 1/20 of the absorbed amount of the anionic cross-linked polymer 3B.

Therefore, the water absorbent resin 3 is configured to be able to absorb a material having a low electrolyte concentration (for example, pigment-based ink containing pigment) and a material having a high electrolyte concentration (for example, a dye-based ink containing a dye), in consideration of the mixing ratio of the nonionic cross-linked polymer 3A and the anionic cross-linked polymer 3B.

Here, in the absorbent composite 10A, when the content of the anionic cross-linked polymer 3B in the water absorbent resin 3 is larger than the content of the nonionic cross-linked polymer 3A, the material having a low electrolyte concentration such as a pigment-based ink containing a pigment can be absorbed more rapidly.

In addition, in the absorbent composite 10A, when the content of the nonionic cross-linked polymer 3A in the water absorbent resin 3 is larger than the content of the anionic cross-linked polymer 3B, even the material having a high electrolyte concentration, such as a dye-based ink containing a dye, can be efficiently absorbed.

In the absorbent composite 10A, the content of the anionic cross-linked polymer 3B in the water absorbent resin 3 is preferably 10% or more and less than 78%. As a result, even the material having a low electrolyte concentration and the material having a high electrolyte concentration can be reliably absorbed.

The anionic cross-linked polymer 3B is (i) unsaturated monocarboxylic acids such as acrylic acid and methacrylic acid, and salts thereof; (ii) unsaturated dicarboxylic acids such as fumaric acid, maleic acid, methylene glutaric acid and itaconic acid, and salts thereof (which may be mono salts or disalts); (iii) unsaturated sulfonic acids such as 3-allyloxy-2-hydroxypropane sulfonic acid, (meth) allyl sulfonic acid and isoprene sulfonic acid, and salts thereof.

The nonionic cross-linked polymer 3A is (iv) unsaturated alcohol such as hydroxyethyl (meth) acrylate, hydroxypropyl (meth) acrylate, 3-(meth) allyloxy-1,2-dihydroxypropane, (meth) allyl alcohol, and isoprenol, and alkylene oxide adducts obtained by adding an alkylene oxide to these hydroxyl groups; (v) (meth) acrylic esters such as methyl (meth) acrylate, ethyl (meth) acrylate, butyl (meth) acrylate, and cyclohexyl (meth) acrylate; (vi) N-substituted or unsubstituted (meth) acrylamides such as (meth) acrylamides, N-monomethyl (meth) acrylamides, N-monoethyl (meth) acrylamides, and N, N-dimethyl (meth) acrylamides; (vii) vinyl aryl monomers such as styrene, indene, and vinyl aniline; (viii) alkenes such as ethylene, propylene, butadiene, isobutylene, and octene; (ix) vinyl carboxylates such as vinyl acetate and vinyl propionate; (x) N, N-dimethylaminoethyl (meth) acrylate, N, N-dimethylaminoethyl (meth) acrylamide, vinylpyridine, vinylimidazole, and unsaturated amine; (xi) vinylamides such as vinylformamide, vinylacetamide, and vinyloxazolidone; (xii) unsaturated anhydrides such as maleic anhydride and itaconic anhydride; (xiii) vinyl ethylene carbonate and derivatives thereof; (xiv) ethyl (meth) acrylate 2-sulfonate and derivatives thereof; (xv) vinyl ethers such as methyl vinyl ether, ethyl vinyl ether, and butyl vinyl ether. Among these, the monomers (i) to (x) are preferable, and the monomers (i), (v), (vi), (vii), (ix), and (x) are more preferable. These may be used only one type and may be used two or more types in combination. Examples of the salt in the above (i) to (iii) and (x) include metal salts, ammonium salts, organic amine salts, and the like. Examples of the alkylene oxide in the above (iv) include ethylene oxide, propylene oxide, and the like, an alkylene oxide having 1 to 20 carbon atoms is preferable, and an alkylene oxide having 1 to 4 carbon atoms is more preferable. The addition mole number of the alkylene oxide in the above (iv) is preferably 0 to 50 moles, and more preferably 0 to 20 moles per mole of the compound of the above (iv).

The nonionic cross-linked polymer 3A and the anionic cross-linked polymer 3B may have any shape, for example, scaly, acicular, fibrous, or particulate shape, and the majority thereof is preferably in the form of particles. When the nonionic cross-linked polymer 3A and the anionic cross-linked polymer 3B are in the form of particles, the permeability of the ink can be easily ensured. In addition, the nonionic cross-linked polymer 3A and the anionic cross-linked polymer 3B can be suitably carried on the fiber base material 2 (fibers). The particulate shape means having an aspect ratio (ratio of maximum length to minimum length) of 0.3 or more and 1.0 or less. The average particle diameter of the particles (“arithmetic mean value of the diameter of the particles” defined in JIS Z 8901: 2006 “Test powder and particles for test”) is preferably 10 μm or more and 800 μm or less, more preferably 15 μm or more and 400 μm or less, and still more preferably 25 μm or more and 150 μm or less. The difference between the average particle diameter of the nonionic cross-linked polymer 3A and the average particle diameter of the anionic cross-linked polymer 3B is preferably in the range of 300 μm or less so as not to cause specific gravity separation when mixed. Furthermore, the range of 200 μm or less is more preferable, and the range of 100 μm or less is still more preferable.

In addition, the paper piece 1 may contain components other than those described above (other components). Examples of such components include surfactants, lubricants, antifoaming agents, fillers, antiblocking agents, ultraviolet absorbers, pigments, colorants such as dyes, flame retardants, flow improvers, and the like.

In addition, as illustrated in FIG. 2, the water absorbent resin 3 (nonionic cross-linked polymer 3A and anionic cross-linked polymer 3B) is in contact with one surface side of each of the fiber base materials 2, and a portion thereof penetrates in the inner side from one surface of the fiber base material 2. That is, a portion of the water absorbent resin 3 is impregnated in the fiber base material 2. As a result, the carrying capacity of the water absorbent resin 3 to the fiber base material 2 can be enhanced. Therefore, the water absorbent resin 3 can be prevented from detaching in the container 9. As a result, the absorption characteristics of the ink can be exhibited over a long period of time, and the water absorbent resin 3 can be prevented from being unevenly distributed in the container 9. As a result, it is possible to prevent the occurrence of unevenness in the absorption characteristics of the ink.

In the present specification, “impregnation” refers to a state of being embedded (in a state of being embedded) in which at least a portion of the particles of the water absorbent resin 3 penetrates in the inner side from the surface of the fiber base material 2. In addition, it is not necessary for all particles to be impregnated. In addition, a state where the particles of the water absorbent resin 3 penetrate in the inside of the fiber base material 2 by softening and come out to the rear surface of the fiber base material 2 is also included.

As illustrated in FIG. 1, each of the paper pieces 1 is preferably in the form of a flexible and elongated (strip) piece. As a result, each of the paper pieces 1 is likely to be deformed. When applied as an ink absorbing material 10 (refer to FIG. 3) as an aggregate including a plurality of paper pieces 1, when stored in the container 9 (refer to FIG. 8) containing the ink absorbing material 10, each of the paper pieces 1 is deformed regardless of the shape of the inside of the container 9, that is, the shape following property is exhibited, and thus the ink absorbing material 10 is collectively stored without difficulty. In addition, the contact area with the ink as the whole of the ink absorbing material 10 can be ensured as much as possible, and thus the absorption performance (absorption characteristics) absorbing the ink is improved.

Although the total length (length in the long side direction) of the paper piece 1 depends on the shape and size of the container 9 to be applied, for example, the total length is preferably 0.5 mm or more and 200 mm or less, more preferably 1 mm or more and 100 mm or less, and still more preferably 2 mm or more and 30 mm or less.

In addition, although the width (length in the short side direction) of the paper piece 1 also depends on the shape and size of the container 9 to be applied, for example, the width is preferably 0.1 mm or more and 100 mm or less, more preferably 0.3 mm or more and 50 mm or less, and still more preferably 1 mm or more and 20 mm or less.

In addition, the aspect ratio of the total length to the width is preferably 1 or more and 200 or less, and more preferably 1 or more and 30 or less. The thickness of the paper piece 1 is also preferably 0.05 mm or more and 2 mm or less, and more preferably 0.1 mm or more and 1 mm or less, for example.

With the above numerical range, it is possible to more suitably carry the water absorbent resin 3, hold the ink by the fibers, and feed the ink into the water absorbent resin 3, and it is possible to make the ink absorption characteristics of the entire paper piece 1 more excellent. Furthermore, the entire ink absorbing material 10 is likely to be deformed, and the shape following property to the container 9 is excellent.

The ink absorbing material 10 may include paper pieces 1 having different sizes and shapes.

In addition, the ink absorbing material 10 may include paper pieces 1 in which at least one of the total length, width, aspect ratio, and thickness are the same as each other, or may include different paper pieces 1 of all of these.

The content of the paper piece 1 having a maximum width of 3 mm or less in the ink absorbing material 10 (absorbent composite 10A) is preferably 30% by weight or more and 90% by weight or less, and more preferably 40% by weight or more and 80% by weight or less. As a result, the occurrence of unevenness in the absorption characteristics of the ink can be more effectively prevented.

If the content of the paper piece 1 having a maximum width of 2 mm or less is too small, when the ink absorbing material 10 is stored in the container 9, a gap is likely to be formed between the paper pieces 1 and there is a concern that unevenness occurs in the absorption characteristics of the ink in the container 9. On the other hand, when the content of the paper piece 1 having a maximum width of 2 mm or less is too high, it tends to be unlikely to form a gap between the paper pieces 1, and it is unlikely to adjust the bulk density of the ink absorbing material 10.

In addition, it is preferable that the paper piece 1 has a regular shape. That is, it is preferable that the paper piece 1 is cut into a regular shape by a shredder or the like. As a result, unevenness in the bulk density of the ink absorbing material 10 is unlikely to occur, and unevenness in the absorption characteristics of the ink can be prevented in the container 9. In addition, the paper piece 1 cut into a regular shape can reduce the area of the cut surface as much as possible. Therefore, it is possible to suppress dust generation (scattering of fibers and the water absorbent resin 3 and detachment of the water absorbent resin 3) while ensuring appropriate ink absorption characteristics.

The “regular shape” refers to, for example, a shape such as a rectangle, a square, a triangle, a polygon such as a pentagon, a circle, an ellipse or the like. In addition, each of the paper pieces 1 may have the same size or a similar shape. In addition, for example, in the case of the rectangle, even if the lengths of the sides are different from each other, if it is a category of the rectangle, it has the regular shape (the same applies to other shapes).

The content of the paper piece 1 having such a regular shape is preferably 30% by weight or more, more preferably 50% by weight or more, and still more preferably 70% by weight or more of the entire ink absorbing material 10.

In addition, the paper piece 1 may have an irregular shape. As a result, each of the paper pieces 1 is likely to be entangled, and it is likely to maintain the shape of the entire ink absorbing material 10 which can prevent the ink absorbing material 10 from being divided or unevenly distributed. In addition, the paper piece 1 having the irregular shape can increase the area of the cut surface (broken surface) as much as possible, and can further increase the contact area with the ink Q (refer to FIG. 8). Therefore, it contributes to quick absorption of the ink.

The “irregular shape” refers to a shape other than the “regular shape” as described above, such as a shape roughly cut or torn by hand (refer to FIG. 1).

In addition, the ink absorbing material 10 may be a mixture of the paper piece 1 having such a regular shape and paper piece 1 having the irregular shape. As a result, both effects described above can be shared.

As described above, each of the paper pieces 1 is an elongated piece (having a longitudinal direction). The container 9 is filled so that the extension directions of each of the paper pieces 1 differ from each other. That is, the plurality of paper pieces 1 are stored in the container 9 as an aggregate without regularity so that the extension directions of the paper pieces 1 slightly intersect with each other (so as not to be parallel). Furthermore, in other words, each of the paper pieces 1 is stored at random (regardless of regularity) in the two-dimensional direction (for example, bottom portion 91 direction) or the three-dimensional direction (three directions in the storage space 93) in the container 9.

In such a stored state, a gap is likely to be formed between the paper pieces 1. As a result, the ink can pass through the gap, and if the gap is too small, the ink can wet and spread by capillary phenomenon, that is, the liquid permeability of the ink can be ensured. As a result, the ink Q flowing downward in the container 9 is prevented from being blocked in the middle, and thus can penetrate into the rear (bottom portion 91) of the container 9. As a result, each of the paper pieces 1 can absorb the ink without excess or deficiency and hold the ink for a long period of time.

In addition, the ink absorbing material 10 can change the shape freely. Therefore, a desired amount (appropriate amount) can be stored in the container 9 and adjustment of bulk density can be easily performed, for example. As a result, it is possible to prevent the occurrence of unevenness in the absorption characteristics of the ink.

In addition, since each of the paper pieces 1 is stored at random, the entire ink absorbing material 10 has an increased chance of contacting the ink, and thus the absorption performance absorbing the ink is improved. In addition, when the ink absorbing material 10 is stored in the container 9, each of the paper pieces 1 can be randomly put into the container 9, and thus the storing operation can be performed easily and rapidly.

In addition, when the volume of the container 9 (storage space 93) is V1 and the total volume of the ink absorbing material 10 before absorbing the ink Q (before water absorption) is V2, the ratio V2/V1 of V1 to V2 is preferably 0.1 or more and 0.95 or less, and more preferably 0.2 or more and 0.90 or less. As a result, a void 95 is generated in the container 9. Each of the paper pieces 1 expands (swells) after absorbing the ink. The void 95 serves as a buffer when each of the paper pieces 1 is expanded, and thus each of the paper pieces 1 can sufficiently absorb the ink.

In addition, the bulk density of the ink absorbing material 10 is preferably 0.01 g/cm³ or more and 0.5 g/cm³ or less, and more preferably 0.03 g/cm³ or more and 0.3 g/cm³ or less. Among these, the bulk density is particularly preferable 0.05 g/cm³ or more and 0.2 g/cm³ or less. As a result, both the water retention and the permeability of the ink can be achieved.

If the bulk density of the ink absorbing material 10 is too low, the content of the water absorbent resin 3 tends to decrease, and the water retention of the ink may be insufficient. On the other hand, if the bulk density of the ink absorbing material 10 is too high, the gap between the paper pieces 1 cannot be sufficiently ensured, and the permeability of the ink may be insufficient.

In addition, since the paper piece 1 is flexible and can be deformed, the bulk density of the ink absorbing material 10 can be easily and properly adjusted, and the bulk density as described above can be obtained.

Next, a method of manufacturing the absorbent composite 10A will be described.

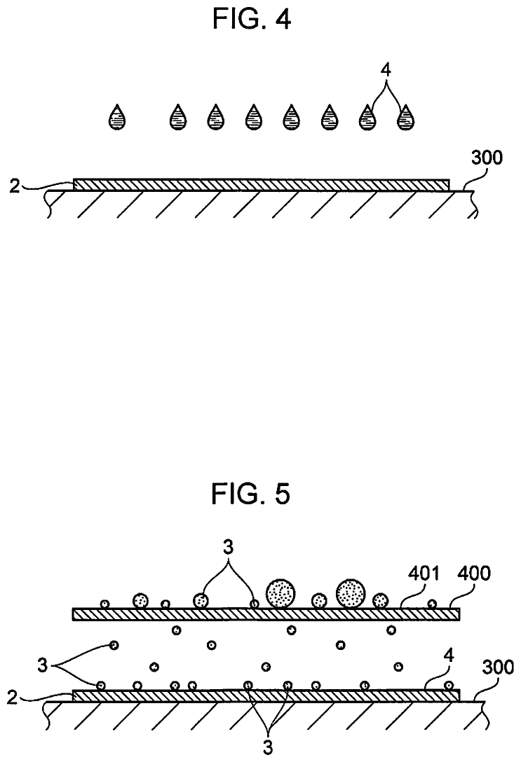

FIGS. 4 to 7 are schematic views illustrating a method of manufacturing the absorbent composite 10A.

First, as illustrated in FIG. 4, the sheet-like fiber base material 2 before being cut into the paper piece 1 is placed on a placement surface of a placement table 300 (placement step).

Water 4 is applied to the sheet-like fiber base material 2 from one surface side (water applying step). Examples of the method of application include application by spray, and a method in which the water 4 is soaked in a sponge roller, and the sponge roller is rolled on one surface of the sheet-like fiber base material 2.

Next, as illustrated in FIG. 5, the water absorbent resin 3 (nonionic cross-linked polymer 3A and anionic cross-linked polymer 3B) is applied onto one surface of the sheet-like fiber base material 2 through a mesh member 400 (water absorbent resin placement step). The mesh member 400 has a mesh 401, of the water absorbent resin 3, the particles larger than the mesh 401 are captured on the mesh member 400, and the particles smaller than the mesh 401 pass through the mesh 401 and are applied onto one surface of the sheet-like fiber base material 2.

As described above, by using the mesh member 400, the particle diameter of the water absorbent resin 3 can be made as uniform as possible. Therefore, it is possible to prevent the occurrence of unevenness in the water absorption by the location of the fiber base material 2.

In addition, the maximum width of the mesh 401 is preferably 0.06 mm or more and 0.15 mm or less, and more preferably 0.08 mm or more and 0.12 mm or less. As a result, the particle diameter of the water absorbent resin 3 applied to the fiber base material 2 can be made to be the particle diameter within the numerical range.

In addition, the shape of the mesh 401 is not particularly limited, and may be any shape such as a triangle, a quadrangle, a polygon of more than these, a circle, or an ellipse. Alternatively, the water absorbent resin 3 classified in advance may be disposed by uniform distribution using a vibrating feeder or the like.

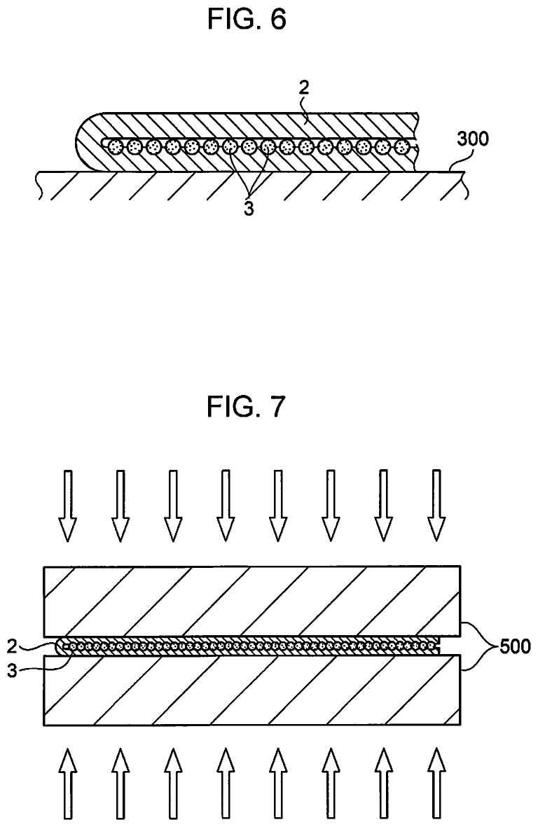

Next, as illustrated in FIG. 6, the sheet-like fiber base material 2 is bent in half so that the surfaces of the fiber base material 2 to which the water absorbent resin 3 is applied are opposed to each other (bending step). The bending unit of the fiber base material 2 is not particularly limited. For example, a suction hole is provided in the placement table 300, and a pump is coupled to the suction hole. In addition, a hinge is provided on the placement table 300 so that the placement table 300 can be folded. The fiber base material 2 is adsorbed to the placement surface of the placement table 300 by driving the pump in a state where the fiber base material 2 is placed on the placement table 300, and the placement table 300 is folded in that state. As a result, the sheet-like fiber base material 2 can be bent in half. The water absorbent resin 3 is in a state of being interposed by the fiber base material 2.

Next, as illustrated in FIG. 7, the bent sheet-like fiber base material 2 is disposed between a pair of heating blocks 500. The pair of heating block 500 is heated and pressurized in a direction where the pair of heating block 500 approaches, and the fiber base material 2 is pressurized in the thickness direction (heating and pressurizing step). As a result, the water absorbent resin 3 is softened by water absorption and heating, and the water absorbent resin 3 penetrates in the inner side of the fiber base material 2 by pressurizing. By releasing the heating and pressurizing, the water is evaporated, and the water absorbent resin 3 adheres to the fiber base material 2 in a state of penetrating in the inner side of the fiber base material 2, and the water absorbent resin 3 is in a state of being impregnated in the fiber base material 2 (refer to FIG. 2).

The pressing force in this step is preferably 0.1 kg/cm² or more and 1.0 kg/cm² or less, and more preferably 0.2 kg/cm² or more and 0.8 kg/cm² or less. In addition, the heating temperature in this step is preferably 80° C. or more and 160° C. or less, and more preferably 100° C. or more and 120° C. or less.

Next, the heated and pressurized sheet-like fiber base material 2 is finely cut, coarse crushed, crushed, or finely cut by hand, for example, with scissors, cutters, mills, shredders, or the like to form the absorbent composite 10A formed of the paper piece 1 (refer to FIG. 1). In addition, the ink absorbing material 10 formed of the plurality of paper pieces 1 is formed (refer to FIG. 3).

An ink absorber 100 is obtained by measuring the ink absorbing material 10 by a desired amount, manually loosening the ink absorbing material 10 to adjust the bulk density, and storing the ink absorbing material 10 in the container 9 (refer to FIG. 8).

Next, a usage aspect of the ink absorbing material 10 will be described. FIG. 8 is a schematic view illustrating an example of the usage aspect of the ink absorbing material 10, and illustrates the configuration of the ink absorber 100 and a printing apparatus 200 provided with the ink absorbing material 10.

In addition, the ink absorber 100 illustrated in FIG. 8 is provided with the ink absorbing material 10 and the container 9 containing the ink absorbing material 10. As a result, the ink absorber 100 can be obtained which exhibits the effects of the ink absorbing material 10 described above.

The printing apparatus 200 (droplet ejecting apparatus) illustrated in FIG. 8 is, for example, an ink jet type color printer. The printing apparatus 200 is provided with a recovery unit 205 that recovers the waste liquid of the ink Q, and the ink absorber 100 is installed in the recovery unit 205. As a result, it is possible to obtain the printing apparatus 200 capable of exhibiting the effects of the ink absorber 100 described above.

The printing apparatus 200 includes an ink ejection head 201 ejecting the ink Q, a capping unit 202 preventing clogging of nozzles 201a of the ink ejection head 201, a tube 203 coupling the capping unit 202 and the ink absorber 100, a roller pump 204 transferring the ink Q from the capping unit 202, and the recovery unit 205.

The ink ejection head 201 has a plurality of nozzles 201a ejecting the ink Q. The ink ejection head 201 can eject the ink Q and perform printing while moving with respect to a recording medium (not illustrated) such as a PPC sheet (refer to ink ejection head 201 indicated by a two-dot chain line in FIG. 8). The nozzle 201a ejecting the ink Q is provided with, for example, a nozzle ejecting a dye-based ink and a nozzle ejecting a pigment-based ink, and as an example, a ratio of the nozzle ejecting the dye-based ink to the nozzle ejecting the pigment-based ink is set to 3 (cyan:yellow:magenta, 1:1:1):3 (black). When suctioned by the capping unit 202, the dye-based ink and the pigment-based ink are mixed.

The capping unit 202 collectively sucks each of the nozzles 201a by the operation of the roller pump 204 when the ink ejection head 201 is in a standby position, and prevents clogging of the nozzles 201a.

The tube 203 is a tube passing the ink Q sucked through the capping unit 202 toward the ink absorber 100. The tube 203 is flexible.

The roller pump 204 is disposed in the middle of the tube 203, and includes a roller portion 204a and a pinching portion 204b which pinches the middle of the tube 203 between the pinching portion 204b and the roller portion 204a. The rotation of the roller portion 204a generates a suction force on the capping unit 202 via the tube 203. The roller portion 204a keeps rotating, so that the ink Q adhering to the nozzle 201a can be fed to the recovery unit 205.

In the recovery unit 205, the ink absorber 100 in which the ink absorbing material 10 is stored is installed. The ink Q is fed into the ink absorber 100 and absorbed by the ink absorbing material 10 in the ink absorber 100 as the waste liquid. The ink Q contains ink of various colors.

As illustrated in FIG. 8, the ink absorber 100 is provided with the ink absorbing material 10, the container 9 storing the ink absorbing material 10, and a lid 8 sealing the container 9.

The ink absorber 100 is detachably attached to the printing apparatus 200, and in the attached state, is used to absorb the waste liquid of the ink Q as described above. As described above, the ink absorber 100 can be used as a so-called “waste liquid tank (waste ink tank)”. When the absorption amount of the ink Q of the ink absorber 100 reaches the limit, the ink absorber 100 can be replaced with a new (unused) ink absorber 100. A detection unit (not illustrated) in the printing apparatus 200 detects whether or not the absorption amount of the ink Q of the ink absorber 100 reaches the limit. In addition, when the absorption amount of the ink Q of the ink absorber 100 reaches the limit, that effect is notified by, for example, a notification unit such as a monitor incorporated in the printing apparatus 200.

The container 9 is a container storing the ink absorbing material 10. The container 9 has a box shape having a bottom portion (bottom plate) 91 having, for example, a rectangular shape in a plan view and four side wall portions 92 erected upward from each side (edge portion) of the bottom portion 91. The ink absorbing material 10 can be stored in a storage space 93 surrounded by the bottom portion 91 and the four side wall portions 92.

The container 9 is not limited to the one having the bottom portion 91 having a square shape in a plan view, may have, for example, the bottom portion 91 having a circular shape in a plan view, and the whole may be cylindrical.

The container 9 is hard, in other words, has a shape-retaining property such that the volume does not change by 10% or more when an internal pressure or an external force acts on the container 9. As a result, the container 9 can maintain the shape of the container 9 itself even if each of the paper pieces 1 of the ink absorbing material 10 absorbs the ink Q, and thereafter expands to receive the force from the paper piece 1 from the inside. Therefore, the installation state of the container 9 in the printing apparatus 200 is stabilized, and each of the paper pieces 1 can stably absorb the ink Q.

The container 9 may be made of a material that does not transmit the ink Q, and although the constituent material is not particularly limited, various resin materials such as cyclic polyolefin and polycarbonate can be used, for example. In addition, as the constituent material of the container 9, various metal materials such as aluminum and stainless steel can be used in addition to the various resin materials, for example.

In addition, the container 9 may be transparent (including translucent) with internal visibility or opaque. In order to reduce the capability of the polymer absorber, it is preferable to be made of a material having a low UV transmittance.

As described above, the ink absorber 100 is provided with the lid 8 sealing the container 9. As illustrated in FIG. 8, the lid 8 has a plate-like shape and can be fitted to an upper opening portion 94 of the container 9. By this fitting, the upper opening portion 94 can be sealed in a liquid tight manner. As a result, for example, when the ink Q is discharged from the tube 203 and dropped, even when the ink Q collides with the ink absorbing material 10 (paper piece 1) and jumps up, the ink Q can be prevented from scattering outward. Therefore, the ink Q can be prevented from adhering to the periphery of the ink absorber 100 and being soiled.

A coupling port 81 to which the tube 203 is coupled is formed at a central portion of the lid 8. The coupling port 81 is configured to include a through-hole which penetrates the lid 8 in the thickness direction. The downstream end portion (lower end portion) of the tube 203 can be inserted into and coupled to the coupling port 81 (through-hole). In addition, at this time, a discharge port (opening portion) 203a of the tube 203 faces downward.

For example, radial ribs or grooves may be formed around the coupling port 81 on the lower surface (rear surface) of the lid 8. The rib or the groove can function as, for example, a regulation portion (guide portion) that regulates the flow direction of the ink Q in the container 9.

In addition, the lid 8 may have an absorbency to absorb the ink Q, or may have a lyophobic property to repel the ink Q.

The thickness of the lid 8 is not particularly limited, and is preferably, for example, 1 mm or more and 20 mm or less, and more preferably 8 mm or more and 10 mm or less. The lid 8 is not limited to a plate-like one having such a numerical range, and may be a film-like (sheet-like) one thinner than the plate-like one. In this case, the thickness of the lid 8 is not particularly limited, and is preferably, for example, 10 μm or more and less than 1 mm.

The ink absorbing material 10 is provided with the plurality of flexible paper pieces 1, and in the present embodiment, the paper pieces 1 are collectively stored in the container 9 and used. As described above, the ink absorber 100 can be attached to the printing apparatus 200 to absorb the ink Q that is the waste liquid.

The number of paper pieces 1 stored in the container 9 is not particularly limited, and the necessary number is selected according to various conditions such as the application of the ink absorber 100, for example. As described above, the ink absorber 100 has a simple configuration in which the necessary number of paper pieces 1 is stored in the container 9. The maximum absorption amount of the ink Q in the ink absorber 100 is adjusted depending on the storage amount of the paper piece 1.

Here, as the ink Q, a pigment-based ink having a low electrolyte concentration or a dye-based ink having a high electrolyte concentration is applied. In addition, an ink in which a pigment and a dye are mixed may be applied to the ink Q. However, since the ink absorbing material 10 is formed of the paper piece 1 having the water absorbent resin 3 in which the nonionic cross-linked polymer 3A and the anionic cross-linked polymer 3B are mixed and the fiber base material 2 containing fibers, any ink can be reliably absorbed regardless of the electrolyte concentration.

As described above, according to this embodiment, the following effects can be obtained.

Since the content of the water absorbent resin 3 in the absorbent composite 10A is appropriate, the contact between the fiber base material 2 and the water absorbent resin 3 is reliably performed. As a result, the water absorbent resin 3 can be prevented from detaching from the fiber base material 2. Therefore, absorption characteristics can be ensured.

In addition, the water absorbent resin 3 contains the nonionic cross-linked polymer 3A and the anionic cross-linked polymer 3B. Therefore, materials having different electrolyte concentrations, for example, both inks of the pigment-based ink and the dye-based ink can be absorbed.

### Second Embodiment

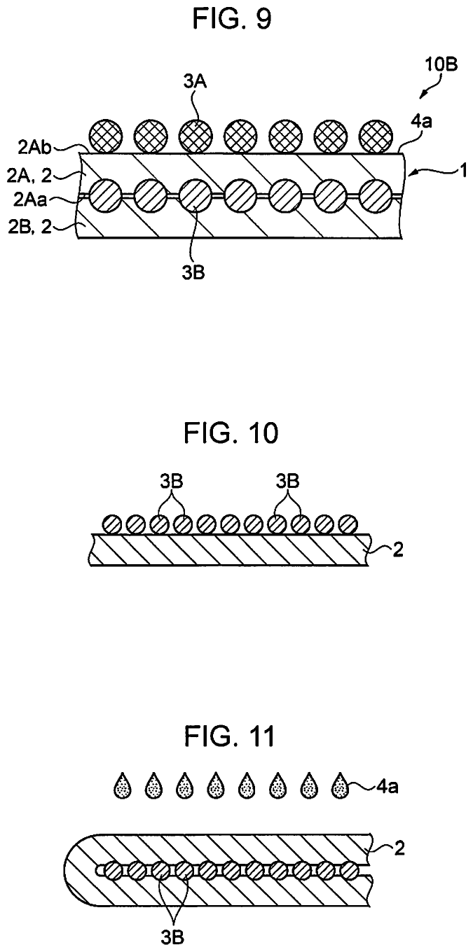

Next, a second embodiment will be described. FIG. 9 is a cross-sectional view illustrating a configuration of an absorbent composite 10B according to the present embodiment.

As illustrated in FIG. 9, in the absorbent composite 10B, the anionic cross-linked polymer 3B is disposed between the two fiber base materials 2 (first fiber base material 2A and second fiber base material 2B). The nonionic cross-linked polymer 3A is disposed on the surface 2Ab of the first fiber base material 2A opposite to a side where the anionic cross-linked polymer 3B is disposed with respect to the first fiber base material 2A.

The configurations of the fiber base material 2, the anionic cross-linked polymer 3B, and the nonionic cross-linked polymer 3A are the same as that in the first embodiment, and thus the description thereof is omitted.

In the absorbent composite 10B of the present embodiment, the anionic cross-linked polymer 3B is disposed between the two fiber base materials 2 (first fiber base material 2A and second fiber base material 2B). The nonionic cross-linked polymer 3A is disposed on one surface 2Ab opposite to the surface 2Aa on the side on which the anionic cross-linked polymer 3B of the first fiber base material 2A is disposed.

The configuration in which the nonionic cross-linked polymer 3A is disposed on one surface of the second fiber base material 2B opposite to the surface on which the anionic cross-linked polymer 3B is disposed may be employed.

In addition, as illustrated in FIG. 9, the anionic cross-linked polymer 3B is in contact with one surface side of each of the fiber base materials 2 (first fiber base material 2A and second fiber base material 2B), and a portion thereof penetrates in the inner side from one surface of the fiber base material 2. That is, a portion of the anionic cross-linked polymer 3B is impregnated in the fiber base material 2. As a result, the carrying capacity of the anionic cross-linked polymer 3B with respect to the fiber base material 2 can be enhanced. Therefore, the anionic cross-linked polymer 3B can be prevented from detaching from the fiber base material 2. As a result, the absorption characteristics of the ink can be exhibited over a long period of time, and the water absorbent resin 3 can be prevented from being unevenly distributed in the container 9. As a result, it is possible to prevent the occurrence of unevenness in the absorption characteristics of the ink (refer to FIG. 8).

The nonionic cross-linked polymer 3A is adhered to the surface 2Ab of the fiber base material 2A by an adhesive 4a. The adhesive 4a is not particularly limited, and a water-soluble adhesive, an organic adhesive or the like can be used. Among these, the water-soluble adhesive is preferable. As a result, even if the water-soluble adhesive is attached to the surface of the water absorbent resin 3 when the ink Q is water-based, the water-soluble adhesive is dissolved when the ink Q contacts the adhesive 4a. Therefore, it is possible to prevent the absorption of the ink Q by the water absorbent resin 3 from being inhibited by the water-soluble adhesive.

Examples of the water-soluble adhesive include proteins such as casein, soybean protein and synthetic protein, various starches such as starch and starch oxide, polyvinyl alcohols containing modified polyvinyl alcohol such as polyvinyl alcohol, cationic polyvinyl alcohol, silyl-modified polyvinyl alcohol, cellulose derivatives such as carboxymethyl cellulose and methyl cellulose, aqueous polyurethane resin, aqueous polyester resin, and the like.

Among these adhesives 4a, it is preferable to use polyvinyl alcohol from the point of surface strength. As a result, the adhesive force of the fiber base material 2 and nonionic cross-linked polymer 3A can sufficiently be raised.

By selecting the type of the adhesive 4a in accordance with the type of the ink Q to be absorbed, the above effect can be exhibited regardless of the type of the ink Q.

The content of the adhesive 4a in the paper piece 1 is preferably 1.0% by weight or more and 70% by weight or less, and more preferably 2.5% by weight or more and 50% by weight or less, based on the fibers. As a result, the effect of containing the adhesive 4a can be obtained more remarkably. When the content of the adhesive 4a is too small, the effect of containing the adhesive 4a cannot be obtained sufficiently. On the other hand, even when the content of the adhesive 4a is too large, the improvement of the carrying capacity of the nonionic cross-linked polymer 3A cannot be further remarkably obtained.

Next, a method of manufacturing an absorbent composite 10B will be described.

FIGS. 10 to 12 are schematic views illustrating a method of manufacturing the absorbent composite 10B.

First, the sheet-like fiber base material 2 before being cut into the paper piece 1 is placed on the placement table 300 (placement step). The placement unit of the placement step is the same as that in the first embodiment (refer to FIG. 4).

The water 4 is applied to the sheet-like fiber base material 2 from one side (water applying step). The unit that applies water in the water applying step is the same as that in the first embodiment (refer to FIG. 4).

Next, as illustrated in FIG. 10, the anionic cross-linked polymer 3B is applied onto one surface of the sheet-like fiber base material 2 (anionic cross-linked polymer placement step). The unit that applies the anionic cross-linked polymer 3B is the same as that of the water absorbent resin placement step of the first embodiment (refer to FIG. 5).

Next, the sheet-like fiber base material 2 is bent in half so that the surfaces of the fiber base material 2 to which the anionic cross-linked polymer 3B is applied are opposed to each other (bending step). The bending unit in the bending step is the same as that in the first embodiment (refer to FIG. 6). As a result, the anionic cross-linked polymer 3B is in a state of being interposed by the fiber base material 2.

Next, the bent sheet-like fiber base material 2 is heated and pressurized (heating and pressurizing step). The heating and pressurizing unit in the heating and pressurizing step is the same as that in the first embodiment (refer to FIG. 7). As a result, the anionic cross-linked polymer 3B adheres to the fiber base material 2 in a state where the anionic cross-linked polymer 3B penetrates in the inner side of the fiber base material 2, and the fiber base material 2 is impregnated with the anionic cross-linked polymer 3B.

Next, as illustrated in FIG. 11, an adhesive 4a is applied to one surface of the fiber base material 2 opposite to the side on which the anionic cross-linked polymer 3B of the fiber base material 2 is disposed (adhesive application step). Examples of the method of application include application by spraying and a method in which the sponge roller is impregnated with the adhesive 4a and the sponge roller is rolled on one surface of the sheet-like fiber base material 2.

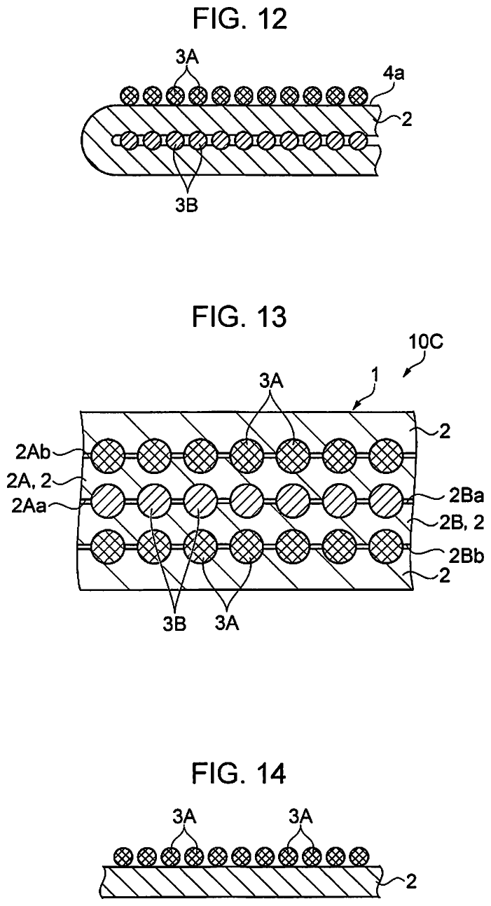

Next, as illustrated in FIG. 12, the nonionic cross-linked polymer 3A is applied to one surface of the fiber base material 2 opposite to the side on which the anionic cross-linked polymer 3B of the fiber base material 2 is disposed. That is, in the adhesive application step, the nonionic cross-linked polymer 3A is applied to the surface of the fiber base material 2 to which the adhesive 4a is applied (nonionic cross-linked polymer placement step). The unit that applies the nonionic cross-linked polymer 3A is the same as that of the water absorbent resin placement step of the first embodiment (refer to FIG. 5). The applied nonionic cross-linked polymer 3A is adhered to the fiber base material 2 by the adhesive 4a. As a result, the carrying capacity of the nonionic cross-linked polymer 3A to the fiber base material 2 can be enhanced, and the nonionic cross-linked polymer 3A can be unlikely to detach from the fiber base material 2. Next, the adhesive 4a is dried. The drying unit dries the adhesive 4a by natural drying by leaving or by a blowing unit using a fan.

Next, the fiber base material 2 is finely cut, coarse crushed, crushed, or finely cut by hand, for example, with scissors, cutters, mills, shredders, or the like to form the absorbent composite 10B formed of the paper piece 1 (refer to FIG. 9). In addition, the ink absorbing material 10 formed of the plurality of paper pieces 1 is formed.

In addition, in the same manner as in the first embodiment, the ink absorber 100 is obtained by measuring the ink absorbing material 10 by a desired amount, manually loosening the ink absorbing material 10 to adjust the bulk density, and storing the ink absorbing material 10 in the container 9 (refer to FIG. 8).

As described above, according to the present embodiment, in addition to the above effects, the following effects can be obtained.

The nonionic cross-linked polymer 3A is disposed on one surface 2Ab of the fiber base material 2 opposite to the side on which the anionic cross-linked polymer 3B is disposed. As a result, for example, when the ink is in contact with the absorbent composite 10B, the ink is in contact with the nonionic cross-linked polymer 3A previously disposed on the surface side. Therefore, first, the ink is absorbed by the nonionic cross-linked polymer 3A, and the electrolytic mass can be reduced. The water whose electrolytic mass is reduced is absorbed by the anionic cross-linked polymer 3B. As a result, the water absorption efficiency can be enhanced.

### Third Embodiment

Next, a third embodiment will be described. FIG. 13 is a cross-sectional view illustrating a configuration of an absorbent composite 10C according to the present embodiment.

As illustrated in FIG. 13, in the absorbent composite 10C, the anionic cross-linked polymer 3B is disposed between the two fiber base materials 2 (first fiber base material 2A and second fiber base material 2B). The nonionic cross-linked polymer 3A is disposed on the surface of the first fiber base material 2A opposite to a side where the anionic cross-linked polymer 3B is disposed. Furthermore, the nonionic cross-linked polymer 3A is disposed on the surface of the second fiber base material 2B.

The configurations of the fiber base material 2, the anionic cross-linked polymer 3B, and the nonionic cross-linked polymer 3A are the same as that in the first embodiment, and thus the description thereof is omitted.

In the absorbent composite 10C of the present embodiment, the anionic cross-linked polymer 3B is disposed between the two fiber base materials 2 (first fiber base material 2A and second fiber base material 2B). The nonionic cross-linked polymer 3A is disposed on one surface 2Ab opposite to the surface 2Aa on the side on which the anionic cross-linked polymer 3B of the first fiber base material 2A is disposed. Furthermore, in the absorbent composite 10C, the nonionic cross-linked polymer 3A is also disposed on one surface 2Bb (the other surface of the fiber base material 2) opposite to the surface 2Ba on the side on which the anionic cross-linked polymer 3B of the second fiber base material 2B is disposed.

In addition, as illustrated in FIG. 13, the anionic cross-linked polymer 3B is in contact with one surface side of each of the fiber base materials 2, and a portion thereof penetrates in the inner side from one surface of the fiber base material 2. That is, a portion of the anionic cross-linked polymer 3B is impregnated in the fiber base material 2. As a result, the carrying capacity of the anionic cross-linked polymer 3B with respect to the fiber base material 2 can be enhanced. Therefore, the anionic cross-linked polymer 3B can be prevented from detaching from the fiber base material 2.

In addition, as illustrated in FIG. 13, the nonionic cross-linked polymer 3A is similarly in contact with one surface side of each of the fiber base materials 2, and a portion thereof penetrates in the inner side from one surface of the fiber base material 2. That is, a portion of the nonionic cross-linked polymer 3A is impregnated in the fiber base material 2. As a result, the carrying capacity of the nonionic cross-linked polymer 3A with respect to the fiber base material 2 can be enhanced. Therefore, the nonionic cross-linked polymer 3A can be prevented from detaching from the fiber base material 2.

According to the above configuration, the absorption characteristics of the ink can be exhibited over a long period of time, and the water absorbent resin 3 can be prevented from being unevenly distributed in the container 9. As a result, it is possible to prevent the occurrence of unevenness in the absorption characteristics of the ink (refer to FIG. 8).

In addition, in the absorbent composite 10C, the amount of the nonionic cross-linked polymer 3A disposed on one of the surface 2Ab of the first fiber base material 2A and the surface 2Bb of the second fiber base material 2B is greater than the amount of the anionic cross-linked polymer 3B disposed between the first fiber base material 2A and the second fiber base material 2B. As a result, it is possible to enhance the water absorption characteristics particularly for the material with high electrolyte concentration. The first fiber base material 2A and the second fiber base material 2B interposing the anionic cross-linked polymer 3B may contain, for example, an ion exchange resin or a coagulant to reduce the electrolytic mass. Alternatively, for example, an ion exchange resin or a coagulant to reduce the electrolytic mass may be disposed on the surface of the first fiber base material 2A and the second fiber base material 2B on which the nonionic cross-linked polymers 3A are disposed.

Next, a method of manufacturing an absorbent composite 10C will be described.

FIGS. 14 to 17 are schematic views illustrating a method of manufacturing the absorbent composite 10C.

First, the sheet-like fiber base material 2 before being cut into the paper piece 1 is placed on the placement table 300 (placement step). The placement unit of the placement step is the same as that in the first embodiment (refer to FIG. 4).

The water 4 is applied to the sheet-like fiber base material 2 from one surface side (water applying step). The unit that applies water in the water applying step is the same as that in the first embodiment (refer to FIG. 4).

Next, as illustrated in FIG. 14, the nonionic cross-linked polymer 3A is applied onto one surface of the sheet-like fiber base material 2 (nonionic cross-linked polymer placement step). The unit that applies the nonionic cross-linked polymer 3A is the same as that of the water absorbent resin placement step of the first embodiment (refer to FIG. 5).

Next, the sheet-like fiber base material 2 is bent in half so that the surfaces of the fiber base material 2 to which the nonionic cross-linked polymer 3A is applied are opposed to each other (first bending step). The bending unit in the bending step is the same as that in the first embodiment (refer to FIG. 6). The nonionic cross-linked polymer 3A is in a state of being interposed by the fiber base material 2.

Next, the bent sheet-like fiber base material 2 is heated and pressurized (first heating and pressurizing step). The heating and pressurizing unit in the first heating and pressurizing step is the same as that in the first embodiment (refer to FIG. 7). As a result, the nonionic cross-linked polymer 3A adheres to the fiber base material 2 in a state where the nonionic cross-linked polymer 3A penetrates in the inner side of the fiber base material 2, and the fiber base material 2 is impregnated with the nonionic cross-linked polymer 3A.

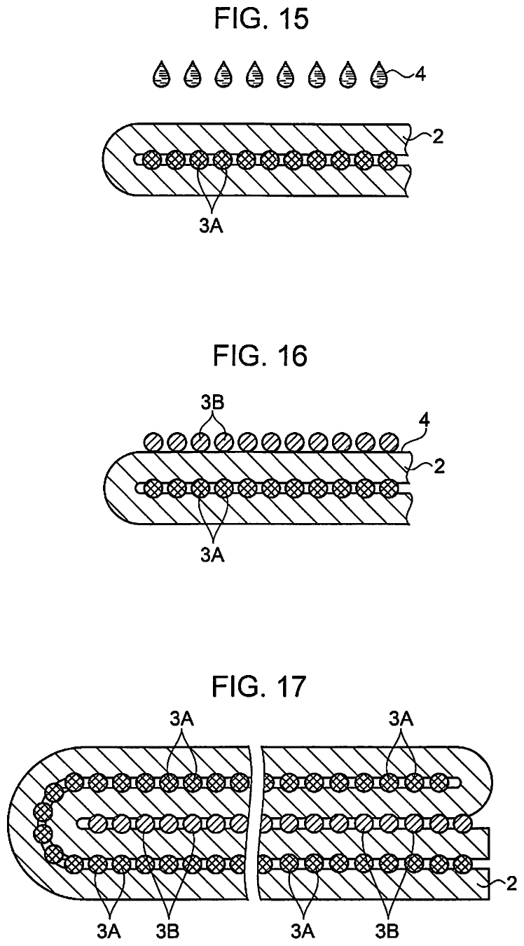

Next, as illustrated in FIG. 15, water 4 is applied to one surface of the fiber base material 2 opposite to the side on which the nonionic cross-linked polymer 3A of the fiber base material 2 is disposed. The unit that applies water is the same as that in the first embodiment (refer to FIG. 4). The same effect can be obtained by applying the adhesive 4a.

Next, as illustrated in FIG. 16, the anionic cross-linked polymer 3B is applied to one surface of the fiber base material 2 opposite to the side on which the nonionic cross-linked polymer 3A of the fiber base material 2 is disposed. That is, the anionic cross-linked polymer 3B is applied to the surface of the fiber base material 2 to which water is applied (anionic cross-linked polymer placement step). The unit that applies the anionic cross-linked polymer 3B is the same as that of the water absorbent resin placement step of the first embodiment (refer to FIG. 5). The applied anionic cross-linked polymer 3B is adhered to the fiber base material 2 by water. As a result, the carrying capacity of the anionic cross-linked polymer 3B to the fiber base material 2 can be enhanced, and the anionic cross-linked polymer 3B can be unlikely to detach from the fiber base material 2.

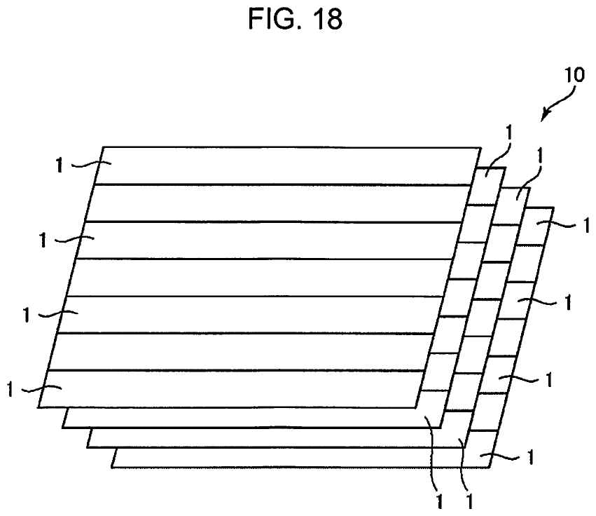

Next, the sheet-like fiber base material 2 is further bent in half so that the vertical direction of the anionic cross-linked polymer 3B is interposed by the surface of the fiber base material 2 adhered to the anionic cross-linked polymer 3B in FIG. 17 (second bending step). The bending unit in the second bending step is the same as that in the first embodiment (refer to FIG. 6). The anionic cross-linked polymer 3B is in a state of being interposed by the fiber base material 2.

Next, the bent sheet-like fiber base material 2 is heated and pressurized (second heating and pressurizing step). The heating and pressurizing unit in the second heating and pressurizing step is the same as that in the first embodiment (refer to FIG. 7). As a result, the anionic cross-linked polymer 3B adheres to the fiber base material 2 in a state where the anionic cross-linked polymer 3B penetrates in the inner side of the fiber base material 2, and the fiber base material 2 is impregnated with the anionic cross-linked polymer 3B.

Next, the fiber base material 2 is finely cut, coarse crushed, crushed, or finely cut by hand, for example, with scissors, cutters, mills, shredders, or the like to form the absorbent composite 10C formed of the paper piece 1 (refer to FIG. 13). In addition, the ink absorbing material 10 formed of the plurality of paper pieces 1 is formed.

In addition, in the same manner as in the first embodiment, the ink absorber 100 is obtained by measuring the ink absorbing material 10 by a desired amount, manually loosening the ink absorbing material 10 to adjust the bulk density, and storing the ink absorbing material 10 in the container 9 (refer to FIG. 8).

As described above, according to the present embodiment, in addition to the above effects, the following effects can be obtained.

The absorbent composite 10C has a configuration in which the nonionic cross-linked polymer 3A is disposed on both surfaces of the fiber base material 2, so that the water absorption efficiency can be further enhanced. Since the nonionic cross-linked polymer 3A absorbs even a material having a high electrolyte concentration, the nonionic cross-linked polymer 3A provided on the outside contacts the ink first to absorb the ink, and the remaining liquid reaches the anionic cross-linked polymer 3B provided in the central portion, so that the ink can be absorbed efficiently.

### Fourth Embodiment

Next, a fourth embodiment will be described. FIG. 18 is a schematic view illustrating an example of an aspect of the ink absorbing material 10. In FIG. 18, it is the same as the first embodiment except that a storage state of the paper piece 1 in the container 9 is different.

As illustrated in FIG. 18, each of the paper pieces 1 is an elongated piece (having a longitudinal direction). In the container 9, the plurality of paper pieces 1 are stored in a state where the extension directions of the paper pieces 1 are aligned in the left-right direction (predetermined one direction) in FIG. 18. That is, each of the paper pieces 1 is regularly disposed side by side in the container 9. In addition, the paper pieces 1 overlapped each other are also contained. Such a storage state of the paper piece 1 is an effective configuration when the flow-down speed (penetration speed) of the ink is to be delayed, for example, when the ink Q flows down toward the bottom portion 91 in the container 9.

The ink absorbing material 10 includes a plurality of regularly disposed paper pieces 1, and in addition, for example, may include a plurality of randomly disposed paper pieces 1 as described in the first embodiment.

### Fifth Embodiment

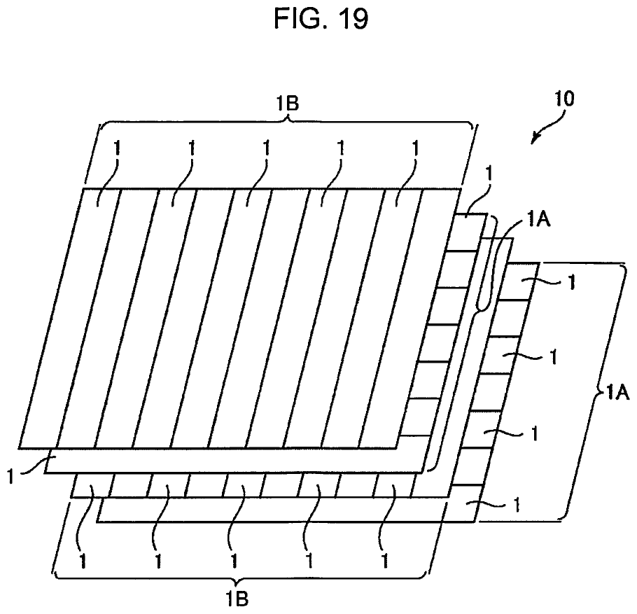

Next, a fifth embodiment will be described. FIG. 19 is a schematic view illustrating an example of an aspect of the ink absorbing material 10.

The present embodiment is the same as the fourth embodiment except that a storage state of the paper piece 1 in the container is different.

As illustrated in FIG. 19, in the container 9, the ink absorbing material 10 includes a first paper piece group 1A in which the extension direction is aligned in the left-right direction in FIG. 19, and a second paper piece group 1B in which the extension direction is aligned in a direction from diagonally upper right to diagonally lower left in FIG. 19. That is, the extension direction of the paper piece 1 of the first paper piece group 1A and the extension direction of the paper piece 1 of the second paper piece group 1B intersect (orthogonal) with each other. In addition, the first paper piece group 1A and the second paper piece group 1B are alternately overlapped each other. Such a storage state of the paper piece 1 is effective configuration when the flow-down speed of the ink Q is to be further delayed than that in the fourth embodiment, for example.

### Sixth Embodiment

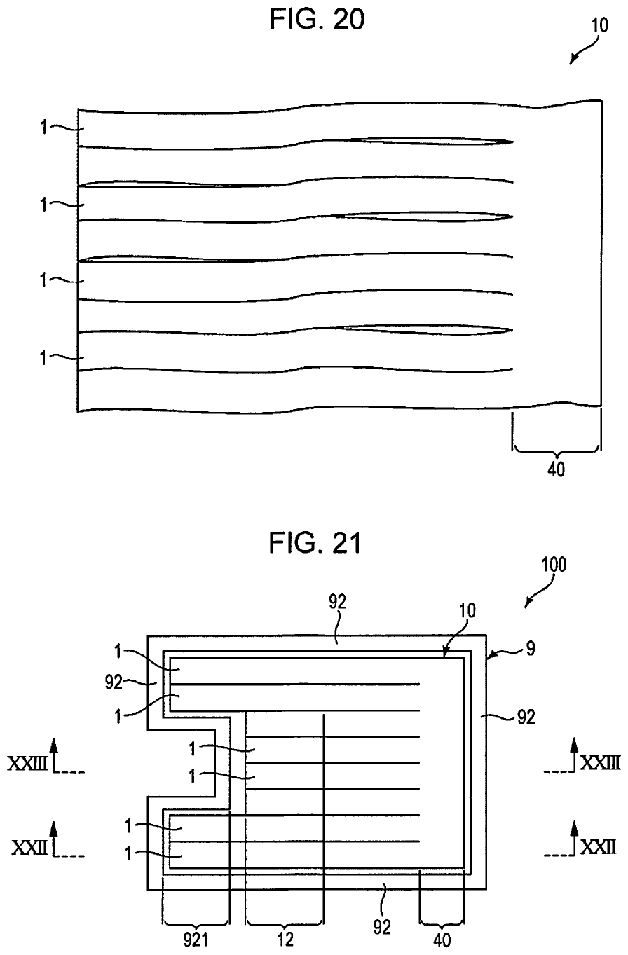

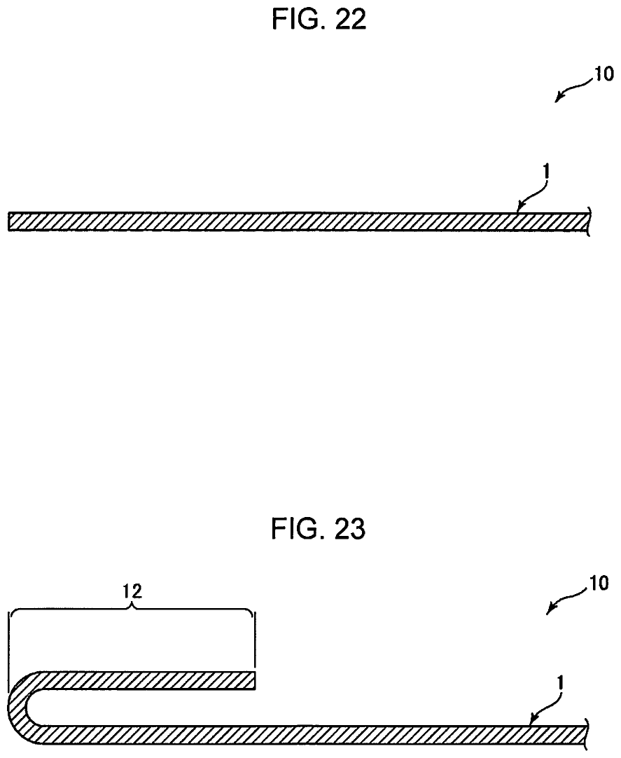

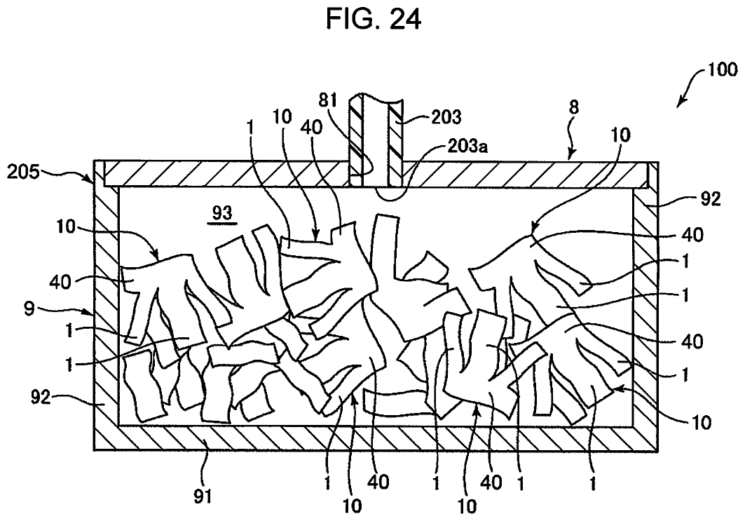

FIG. 20 is a schematic view illustrating an example of an aspect of the ink absorbing material 10. FIG. 21 is a plan view illustrating a state of the ink absorbing material 10 illustrated in FIG. 20 in the container 9. FIG. 22 is a cross-sectional view taken along line XXII-XXII in FIG. 21. FIG. 23 is a cross-sectional view taken along line XXIII-XXIII in FIG. 21. FIG. 24 is a schematic view illustrating an example of a storage aspect of the ink absorbing material 10 stored in the ink absorber 100.

Hereinafter, the ink absorbing material 10, the ink absorber 100, and the printing apparatus 200 of the present disclosure will be described with reference to these drawings, the differences from the above-described embodiment will be mainly described, and the same matters will not be described.

The present embodiment is the same as the first embodiment except that the configuration of the ink absorbing material 10 is different.

As illustrated in FIG. 20, in the present embodiment, the ink absorbing material 10 is provided with a connection portion 40 connecting a plurality of paper pieces 1 (especially, end portions). As a result, when the ink absorbing material 10 is stored in the container 9, the connection portion 40 can be gripped and the plurality of paper pieces 1 for the connection portion 40 can be collectively stored in the container 9. Therefore, the operation of storing the ink absorbing material 10 in the container 9 can be performed easily and rapidly.

Similarly to the paper piece 1, the connection portion 40 also preferably includes the fiber base material 2 containing fibers and the water absorbent resin 3 carried on the fiber base material 2. That is, a plurality of parallel cuts (cuts) are made from one end side to the other end side of one sheet of paper material (sheet), and the connection portion 40 can be obtained by stopping the cut in the middle (before reaching the other end). The plurality of paper pieces 1 form the ink absorbing material 10 by the end portions on the other end side being continuously connected in the short direction of each of the paper pieces 1. That is, the position of the connection portion 40 is located on the side opposite to the side where the cut is inserted. In addition, the connection portion 40 may be formed of another member, such as a paper tape, a stapler, another binding member or the like, for example.

In addition, although the number of sheets of the paper piece 1 connected via the connection portion 40 is eight sheets in this embodiment, when the number of sheets is two or more sheets, the number of sheets will not be limited thereto.

In addition, the connection portion 40 is not limited to a portion connecting the end portions of the other end side of each of the paper pieces 1 to each other. For example, the connection portion 40 may connect the middle in the longitudinal direction of each of the paper pieces 1 (a portion of each of the paper pieces 1). Also in this case, the operation of storing the ink absorbing material 10 in the container 9 can be performed easily and rapidly.

In addition, in the container 9, one sheet (ink absorbing material 10) connected via the connection portion 40 may be stored one by one, or a plurality of stacked ones may be stored.

In addition, in the container 9, the plurality of paper pieces 1 may be stored separately (independently), respectively. In this case, a plurality of randomly disposed paper pieces 1 as described in the first embodiment may be included, or a plurality of regularly disposed paper pieces 1 as described in the fourth embodiment may be included.

As illustrated in FIG. 21, in the present embodiment, the container 9 has a projecting portion 921 projecting (protruded) toward the inner side on one side wall portion 92 of the four side wall portions 92. A side opposite to the projecting portion 921 is recessed and is, for example, a relief portion that prevents interference with members around when the ink absorber 100 is installed in the printing apparatus 200.

The projecting portion 921 is formed in one side wall portion 92 of one of the four side wall portions 92 in the present embodiment, and is not limited thereto. The projecting portions 921 may be formed, for example, in two, three or four (all) side wall portions 92.

As described above, each of the paper pieces 1 is an elongated piece. In the container 9, the bent paper piece 1 is included in these paper pieces 1. That is, among the plurality of paper pieces 1, there is one having a bent portion 12 in which an end portion opposite to the connection portion 40 is bent (refer to FIGS. 21 and 23). The length of the paper piece 1 in the container 9 is adjusted by the bent portion 12 and interference with the projecting portion 921 is prevented. As a result, the ink absorbing material 10 can be easily stored in the container 9. In addition, the storage state of the ink absorbing material 10 thereafter is also stabilized. In addition, the thickness of the paper piece 1 having the bent portion 12 in the container 9 is also increased (adjusted) by the amount of the bent portion.

The paper piece 1 other than the paper piece 1 having the bent portion 12 has an end portion on the side opposite to the connection portion 40 extended (refer to FIG. 22).

In addition, in the container 9 illustrated in FIG. 24, a plurality of ink absorbing materials 10 provided with a connection portion 40 connecting a plurality of paper pieces 1 are stored, and these are randomly stored in a two-dimensional direction or a three-dimensional direction. In addition, in each of the ink absorbing materials 10, the paper piece 1 may be bent or twisted, that is, may be deformed into a desired shape. The ink Q can be absorbed rapidly by this storage state as well.

### Seventh Embodiment

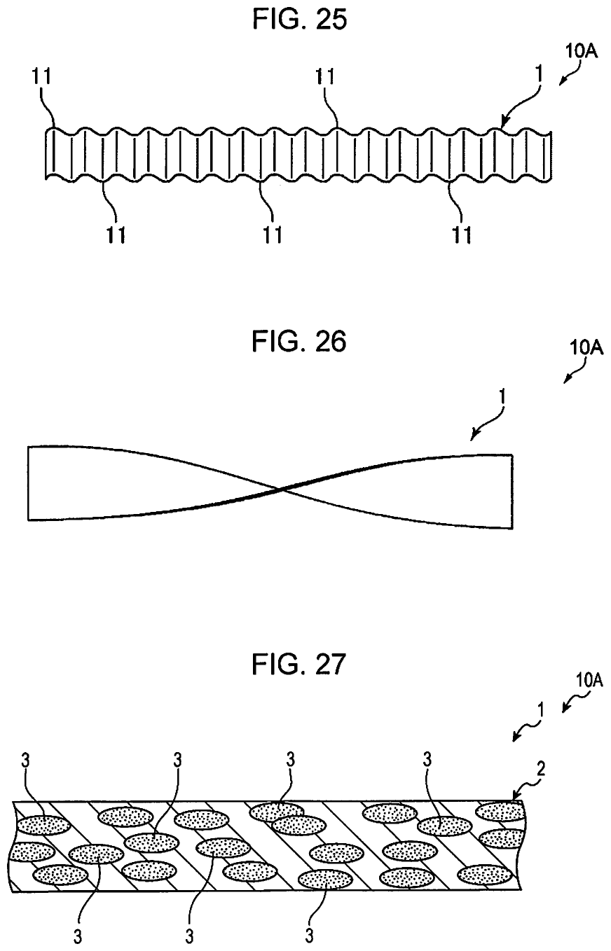

FIG. 25 is a schematic view illustrating another configuration of the absorbent composite 10A.

The present embodiment is the same as the first embodiment except that the shape of the paper piece 1 of the absorbent composite 10A is different.

As illustrated in FIG. 25, in the present embodiment, the paper piece 1 has a bent portion (fold) 11 that is bent (or curved) in the opposite direction alternately a plurality of times along the longitudinal direction. That is, the paper piece 1 has a corrugated shape. The paper piece 1 deformed in this manner is obtained, for example, by bulky processing (bulk processing). As a result, the paper pieces 1 become bulky, and thus increasing the chances that the paper pieces 1 per sheet come into contact with the ink Q. As a result, the ink Q can be absorbed as much as possible.

The bent portion 11 may be alternately bent in the opposite direction in the longitudinal direction of the paper piece 1 and may be a fold along the width direction.

In addition, the number of the bent portions 11 is not limited to a plurality, and may be one.

### Eighth Embodiment

FIG. 26 is a schematic view illustrating another configuration of the absorbent composite 10A.

The present embodiment is the same as the first embodiment except that the shape of the paper piece 1 of the absorbent composite 10A is different.

As illustrated in FIG. 26, in the present embodiment, the paper piece 1 is twisted at least once in the middle in the longitudinal direction. As a result, the paper pieces 1 become bulky, and thus increasing the chances that the paper pieces 1 per sheet come into contact with the ink Q. As a result, the ink Q can be absorbed as much as possible.

In addition, the twist and bent portion 11 described above may be mixed in one paper piece 1.

### Ninth Embodiment

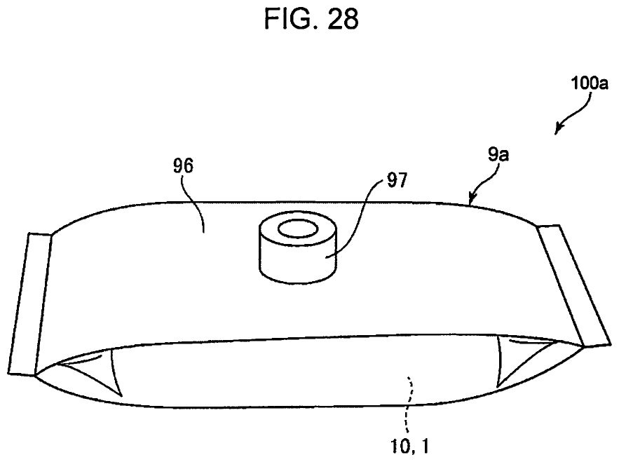

FIG. 27 is a schematic view illustrating another configuration of the absorbent composite 10A.

The present embodiment is the same as the first embodiment except that a positional relationship between the fiber base material 2 and the water absorbent resin 3 is different.

As illustrated in FIG. 27, in the present embodiment, the water absorbent resin 3 in the fiber base material 2 is present in the middle in the thickness direction of the fiber base material 2. That is, the water absorbent resin 3 is impregnated in the thickness direction of the fiber base material 2. As a result, the ink Q can be held (absorbed) on the central portion side in the thickness direction of the paper piece 1 as much as possible, and thus the holding state of the ink Q can be maintained for a long period time. In addition, the water absorbent resin 3 can also be prevented from detaching from the fiber base material 2.

### Tenth Embodiment

FIG. 28 is a perspective view illustrating a configuration of another ink absorber 100a.

The present embodiment is the same as the first embodiment except that the configuration of a container 9a of the ink absorber 100a is different.

As illustrated in FIG. 28, in the present embodiment, the container 9a is flexible, that is, in the form of a flexible bag. In other words, the container 9a has a shape-retaining property such that the volume V1 changes by 10% or more when an internal pressure or an external force acts on the container 9a. In FIG. 28, as an example, the ink absorber 100a is a “pillow package”. Such a container 9a can be appropriately deformed according to the installation location of the ink absorber 100a. As a result, the installation state of the ink absorber 100a is stabilized, and each of the paper pieces 1 (ink absorbing material 10) can stably absorb the ink Q. In addition, even if each of the paper pieces 1 absorbs the ink Q and expands, the container 9a can be deformed following the expansion. In addition, it also contributes to reducing the weight of the ink absorber 100a (container 9a).

In addition, a coupling port 97 to which the tube 203 is coupled is provided in the central portion on the side of the upper surface 96 of the container 9a. The coupling port 97 is tubular and protrudes upward.

The constituent material of the container 9a is not particularly limited, and examples thereof include various thermoplastic elastomers such as polyolefin such as polyethylene and ethylene-vinyl acetate copolymer (EVA), polyesters such as polyethylene terephthalate (PET) and polybutylene terephthalate (PBT), polyurethane, and the like.

Although the absorbent composite 10A, the ink absorbing material 10, the ink absorbers 100 and 100a, and the printing apparatus 200 of the present disclosure is described above with reference to the illustrated embodiment, the present disclosure is not limited thereto. Each parts constituting the ink absorbing material 10, the ink absorbers 100 and 100a, and the printing apparatus 200 can be replaced with any configuration capable of performing the same function. In addition, any component may be added.

In addition, the absorbent composite 10A, the ink absorbing material 10, the ink absorbers 100 and 100a, and the printing apparatus 200 according to the present disclosure may be a combination of any two or more configurations (features) of the respective embodiments.

In addition, as the application of the ink absorber 100 and 100a of the present disclosure, although it is “waste liquid tank (waste ink tank)” in each embodiment, it is not limited thereto. For example, it may be an “ink leak receiver” that absorbs ink that is inadvertently leaked from the ink flow path of the printing apparatus 200.

Hereinbefore, in the embodiment, although the ink absorbing material 10 provided with the plurality of paper pieces 1 of the absorbent composite 10A is described, it is not limited thereto. For example, it may be a deodorant provided with the plurality of paper pieces 1 of the absorbent composite 10A.

The deodorant includes N-vinyl lactam-based cross-linked polymer or N-vinyl lactam-based cross-linked polymer-containing composition. The odorous components in which the deodorant can exert the effects are not particularly limited, and examples thereof include thiols such as methyl mercaptan, amines such as ammonia, carboxylic acids such as acetic acid, aldehydes such as nonenal, diketones such as diacetyl, and the like. That is, the N-vinyl lactam-based cross-linked polymer of the present disclosure can exert a deodorizing effect on various odorous components, and the reason is considered as follows. The cross-linked polymer of the present disclosure can absorb an odor component by the N site and the carbonyl group possessed by the lactam ring derived from N-vinyl lactam, and the cross-linked polymer of the present disclosure is hygroscopic. Therefore, it is conceivable that water-soluble odorous components can be adsorbed via the absorbed water. The cross-linked polymer of the present disclosure exerts a more excellent deodorizing effect on carboxylic acids such as acetic acid among the above-described odorous components. The form of the deodorant is not particularly limited, and may be any form such as liquid, gel, paste, powder, solid, and the like.

In addition, a deodorizer provided with the deodorant may be used. The deodorizer is not particularly limited, and examples thereof include those having the deodorant of the present disclosure and a vent, those having the deodorant of the present disclosure and a mechanism sucking air, or the like.

The present disclosure is also a method of using the above deodorant by incorporating the deodorant into the deodorizer. Examples of the usage aspect include an aspect in which air to be deodorized is brought into contact with the deodorant of the present disclosure and used. Examples of the contact method include a method of bringing air into contact with the deodorant of the present disclosure by natural convection using the deodorizer provided with the deodorant and the vent of the present disclosure, a method of contacting by supplying sucked air to the deodorant of the present disclosure using the deodorizer provided with the deodorant of the present disclosure and a mechanism sucking air, and the like.

The mechanism sucking air, provided with the above deodorizer is not particularly limited, and examples thereof include a fan, an air pump, and the like.

Examples of the deodorizer provided with the deodorant of the present disclosure and the mechanism sucking air include an air cleaner, an air-conditioner, and the like. When the deodorant of the present disclosure is used in such an air purifier, an air conditioner, and the like, it is preferable to be used in a rotating member such as a fan or a filter.

In addition, a fragrance provided with a plurality of paper pieces 1 of the absorbent composite 10A may be used.

The fragrance contains an N-vinyl lactam-based cross-linked polymer and a fragrance component. The fragrance component is not particularly limited, and is preferably volatile, and examples thereof include perfumes and essential oils.

In addition, cosmetics provided with the plurality of paper pieces 1 of the absorbent composite 10A may be used.

The cosmetics contain an N-vinyl lactam-based cross-linked polymer and/or an N-vinyl lactam-based cross-linked polymer-containing composition. The method of using the N-vinyl lactam-based cross-linked polymer and/or the N-vinyl lactam-based cross-linked polymer-containing composition of the present disclosure as cosmetics is also one of the present disclosure. The above cosmetics may contain other components other than the N-vinyl lactam-based cross-linked polymer and/or the N-vinyl lactam-based cross-linked polymer-containing composition. The other components are not particularly limited, and examples thereof include oil base, moisturizer, feel improver, surfactant, polymer agent, thickener, gelling agent, solvent, propellant, antioxidant, reducing agent, oxidizing agent, preservative, antimicrobial agent, chelating agent, pH adjuster, acids, alkalis, powders, inorganic salts, uv absorber, skin lightening agent, vitamins and derivatives thereof, anti-inflammatory agent, anti-inflammatory agent, drugs for hair growth, circulation promoting agent, stimulant, hormones, anti-wrinkle agent, anti-aging agent, shrinking agent, cooling agent, warming agent, wound healing accelerator, irritation reducing agent, analgesics, cell activator, plant extracts, animal extracts, microbe extracts, antipruritic agent, keratolytic agent, dissolver, antiperspirant, refresher, astringent, enzymes, nucleic acids, fragrance, coloring matter, colorant, dyes, pigments, water, and the like.

The cosmetics of the present disclosure are not particularly limited, and examples thereof include skin cosmetics, skin external preparations, and hair cosmetics. The cross-linked product contained in the cosmetics of the present disclosure can exert effects as a moisturizer, a thickener, an oil absorbing agent, or the like in such cosmetics. The present disclosure is also a moisturizer, a thickener, or an oil absorbing agent containing the cyclic N-vinyl lactam-based cross-linked product of the present disclosure.

The above skin cosmetics are not particularly limited, and examples thereof include basic cosmetics such as lotions, creams, emulsions, and cosmetic liquids; makeup cosmetics such as liquid foundations, foundation emulsions, cheek cosmetics, eye shadows, mascaras, lipsticks; cosmetics for cleansing such as cleansing creams, face washing foams, liquid cleansers; cosmetics such as sunscreen cosmetics (including quasi-drugs); and bath cosmetics such as bath preparations.

Examples of the above external skin preparations include external medicines such as liniment agents, lotions, and ointments.

The hair cosmetics are not particularly limited, and examples thereof include shampoo, rinse, treatment, wax, spray, gel, mist, and the like.

The present disclosure is also the skin cosmetics, the skin external preparations, or the hair cosmetics containing the cross-linked polymer of the present disclosure.

Next, examples of the present disclosure will be described.

### Example 1

### [1] Preparation of Ink Absorbing Material

First, a PPC sheet (recycled cut size G80<toppan foams>) having a 29.7 cm long, 21 cm wide, and 0.1 mm thickness was prepared. The average length of fibers contained in this PPC sheet was 0.71 mm, the average width was 0.2 mm, and the aspect ratio (average length/average width) was 3.56. In addition, the weight of the PPC sheet was 4 g/one sheet.

Next, a small amount of water was sprayed onto this PPC sheet by spraying from one surface side.

Next, a water absorbent resin in which a nonionic cross-linked polymer and an anionic cross-linked polymer were mixed was applied to the surface side of the PPC sheet to which water was sprayed. At this time, the water absorbent resin was applied while passing through a sieve (JTS-200-45-106 manufactured by Tokyo Screen Co., Ltd.) having a mesh having a mesh size of 0.106 mm (refer to FIG. 5).

As the water absorbent resin, particles of an anionic cross-linked polymer: ST-500MPSA (manufactured by Sanyo Chemical Industries, Ltd.) and particles obtained by grinding a nonionic cross-linked polymer: nonionic (alkylene oxide) aqua coke TWB-P (Sumitomo Seika) were used.

Here, the content of the anionic cross-linked polymer in the water absorbent resin was 10%.

The PPC sheet (sheet-like fiber base material) was bent in half so that a valley was formed on the surface to which the water absorbent resin was attached. In this bent state (A5 size), the sheet-like fiber base material was pressurized and heated in the thickness direction using a pair of heating blocks as illustrated in FIG. 7. The pressurizing was performed at 0.3 kg/cm² and the heating temperature was 100° C. In addition, the heating and pressurizing time was 2 minutes.

The heating and pressurizing were released, and when the sheet-like fiber base material reached normal temperature, the sheet-like fiber base material was cut into paper pieces of 2 mm×15 mm. The average particle diameter of the water absorbent resin was 35 to 50 μm. In addition, the average length of the fiber was 25 mm, and the average width of the fiber base material was 10 mm. In addition, in each of the paper pieces, the water absorbent resin was impregnated (embedded) in the fiber base material.

### Example 2

In Example 2, when the water absorbent resin was applied to the surface side of the PPC sheet on which the water was sprayed, the content of the anionic cross-linked polymer in the water absorbent resin was 50%. An ink absorbing material was prepared in the same manner as in Example 1 except for the change in the content.

### Example 3

In Example 3, when the water absorbent resin was applied to the surface side of the PPC sheet to which the water was sprayed, the content of the anionic cross-linked polymer in the water absorbent resin was 77%. An ink absorbing material was prepared in the same manner as in Example 1 except for the change in the content.

### Comparative Example

In the comparative example, when the water absorbent resin was applied to the surface side of the PPC sheet on which the water was sprayed, the content of the anionic cross-linked polymer in the water absorbent resin was 83%. An ink absorbing material was prepared in the same manner as in Example 1 except for the change in the content.

### [2] Evaluation

### [2-1] Absorption Characteristics

First, a plurality of plastic containers of 100 mL of New Disposable Cup manufactured by As One Corporation were prepared, and 2.0 g of the ink absorbing materials manufactured in each of Examples and Comparative Example were placed in different containers, respectively. When the ink absorbing material was confirmed in the state of being placed in the container, detachment of the water absorbent resin could not be substantially confirmed.

Next, 20 cc of a commercially available ink jet ink (manufactured by Seiko Epson Co., Ltd., HSM-C) as a dye-based ink was poured into a container containing the ink absorbing material. Thereafter, the container was inclined at each time of 30 minutes and 24 hours, and the remaining amount of ink in the container was visually observed and evaluated according to the following criteria.

A: The remaining amount of ink is absent.

B: The remaining amount of ink is over 0 cc and less than 5 cc.

C: The remaining amount of ink is 5 cc or more and less than 10 cc.

D: The remaining amount of ink is 10 cc or more.

The results are as illustrated in Table 1.

TABLE 1

| | Ink absorbing material | Evaluation | |
|---|---|---|---|
| | Content of anionic | Absorption | |
| | cross-linked polymer | characteristics | |
| | in water absorbent | After | After |
| | resin (% by weight) | 30 minutes | 24 hours |
| Example 1 | 10 | B | A |
| Example 2 | 50 | B | A |
| Example 3 | 77 | D | A |
| Comparative | 83 | D | D |
| Example | | | |

As illustrated in Table 1, in Examples 1 to 3, the content of the anionic cross-linked polymer in the water absorbent resin was optimized, and excellent absorption characteristics could be confirmed in the evaluation results after 24 hours. On the other hand, in Comparative Example, it was found that the absorption characteristics of the ink were inferior to those in Examples 1 to 3.

The contents derived from the embodiments will be described below.

An absorbent composite includes a water absorbent resin in which a nonionic cross-linked polymer and an anionic cross-linked polymer are mixed, and a fiber base material containing a fiber, and a content of the water absorbent resin in the absorbent composite is 5% or more and less than 65%.

According to this configuration, since the content of the water absorbent resin in the absorbent composite is appropriate, the contact between the fiber base material and the water absorbent resin is reliably performed. As a result, the water absorbent resin can be prevented from detaching from the fiber base material. Therefore, the absorption characteristics can be ensured.

In addition, the water absorbent resin of the absorbent composite contains the nonionic cross-linked polymer and the anionic cross-linked polymer. The anionic cross-linked polymer has a property of efficiently absorbing a material having a low electrolyte concentration (for example, pigment-based ink containing pigment). In addition, the nonionic cross-linked polymer has a property of absorbing a material having a high electrolyte concentration (for example, dye-based ink containing dye) regardless of the electrolyte concentration. Therefore, materials having different electrolyte concentrations, for example, both inks of the pigment-based ink and the dye-based ink can be absorbed.

In the absorbent composite, it is preferable that the content of the anionic cross-linked polymer is greater than that of the nonionic cross-linked polymer in the water absorbent resin.

According to this configuration, a material having a low electrolyte concentration can be rapidly absorbed.

In the absorbent composite, it is preferable that the content of the nonionic cross-linked polymer is greater than that of the anionic cross-linked polymer in the water absorbent resin.

According to this configuration, even a material having a high electrolyte concentration can be efficiently absorbed.

In the absorbent composite, it is preferable that the content of the anionic cross-linked polymer in the water absorbent resin is 10% or more and less than 78%.

According to this configuration, even a material having a low electrolyte concentration and a material having a high electrolyte concentration can be reliably absorbed.

In the absorbent composite, it is preferable that the water absorbent resin is disposed between two fiber base materials (first fiber base material and second fiber base material).

According to this configuration, since the water absorbent resin is disposed between the two fiber base materials, the water absorbent resin can be prevented from detaching from the fiber base material.

In the absorbent composite, it is preferable that the anionic cross-linked polymer is disposed between the first and second fiber base materials, and the nonionic cross-linked polymer is disposed on a surface of the first fiber base material opposite to a side on which the anionic cross-linked polymer is disposed.

According to this configuration, the nonionic cross-linked polymer is disposed on one surface of the fiber base material opposite to the side on which the anionic cross-linked polymer is disposed. As a result, for example, when the ink is in contact with the absorbent composite, the ink is in contact with the nonionic cross-linked polymer previously disposed on the surface side. Therefore, the ink is first absorbed by the nonionic cross-linked polymer, and the electrolytic mass can be reduced. The water having a reduced electrolytic mass is absorbed by the anionic cross-linked polymer. As a result, the water absorption efficiency can be enhanced.

In the absorbent composite, it is preferable that the nonionic cross-linked polymer is disposed on a surface of the second fiber base material opposite to the side on which the anionic cross-linked polymer is disposed.

According to this configuration, the absorbent composite has a configuration in which the nonionic cross-linked polymer is disposed on both surfaces of the fiber base material, so that the water absorption efficiency can be further enhanced.

In the absorbent composite, it is preferable that the amount of the nonionic cross-linked polymer disposed on any one of the surface of the first fiber base material and the surface of the second fiber base material is greater than the amount of anionic cross-linked polymer disposed between the first and second fiber base materials.

According to this configuration, the water absorption characteristics particularly for the material having a high electrolyte concentration can be further enhanced.

The ink absorbing material includes a plurality of the above absorbent composites.

According to this configuration, even the pigment-based ink having a low electrolyte concentration and the dye-based ink having a high electrolyte concentration can be reliably absorbed.

The deodorant includes a plurality of the above absorbent composites.

According to this configuration, it can be applied as the deodorant utilizing water absorption.

A deodorizer includes the above deodorant.

According to this configuration, the deodorant can be used to apply to the deodorizer such as an air purifier.

The cosmetics include a plurality of the above absorbent composites.

According to this configuration, it can be applied as the cosmetics utilizing water absorption.

What is claimed is:

1. An absorbent composite comprising:
a water absorbent resin in which a nonionic cross-linked polymer and an anionic cross-linked polymer are mixed; and
a fiber base material containing a fiber, wherein
a content of the water absorbent resin in the absorbent composite is 5% or more and less than 65%.

2. The absorbent composite according to claim 1, wherein
a content of the anionic cross-linked polymer is greater than that of the nonionic cross-linked polymer in the water absorbent resin.

3. The absorbent composite according to claim 1, wherein
a content of the nonionic cross-linked polymer is greater than that of the anionic cross-linked polymer in the water absorbent resin.

4. The absorbent composite according to claim 1, wherein
a content of the anionic cross-linked polymer in the water absorbent resin is 10% or more and less than 78%.

5. The absorbent composite according to claim 1, wherein
the water absorbent resin is disposed between two fiber base materials in the absorbent composite.

6. The absorbent composite according to claim 1, wherein
the anionic cross-linked polymer is disposed between first and second fiber base materials, and
the nonionic cross-linked polymer is disposed on a surface of the first fiber base material opposite to a side on which the anionic cross-linked polymer is disposed.

7. The absorbent composite according to claim 6, wherein
the nonionic cross-linked polymer is disposed on a surface of the second fiber base material opposite to the side on which the anionic cross-linked polymer is disposed.

8. The absorbent composite according to claim 7, wherein
an amount of the nonionic cross-linked polymer disposed on any one of the surface of the first fiber base material and the surface of the second fiber base material is greater than an amount of the anionic cross-linked polymer disposed between the first and second fiber base materials.

9. An ink absorbing material comprising:
a plurality of the absorbent composites according to claim 1.

10. A deodorant comprising:
a plurality of the absorbent composites according to claim 1.

11. A deodorizer comprising:
the deodorant according to claim 10.

12. Cosmetics comprising:
a plurality of the absorbent composites according to claim 1.
